(19) United States
(12) Patent Application Publication
(10) Pub. No.: US 2018/0207163 A1
(43) Pub. Date: Jul. 26, 2018

(54) Methods and compositions for treating solid tumors and other malignancies

(71) Applicant: Novartis AG, Basel (CH)

(72) Inventors: Maria Borland, Waltham, MA (US); Christopher Thomas Brain, Cambridge, MA (US); Shivang Doshi, Brookline, MA (US); Sunkyu Kim, Cambridge, MA (US); Jianguo Ma, Newton, MA (US); Josh Murtie, Winchester, MA (US); Hong Zhang, Cambridge, MA (US)

(21) Appl. No.: 15/935,294

(22) Filed: Mar. 26, 2018

Related U.S. Application Data
(63) Continuation of application No. 14/800,893, filed on Jul. 16, 2015, now abandoned.
Application No. 14/800,893 is a continuation of application No. 13/640,863, filed on Oct. 12, 2012, now abandoned.
(60) Provisional application No. 61/323,541, filed on Apr. 13, 2010.

(51) Int. Cl. A61K 31/519; A61K 31/436; A61K 45/06

(52) U.S. Cl. CPC A61K 31/519; A61K 45/06; A61K 31/436

(57) ABSTRACT

A combination of a CDK4/6 inhibitor and an mTOR inhibitor for the treatment of cancer.

### FIELD OF THE INVENTION

A combination of a mammalian target of rapamycin (mTOR) inhibitor and a cyclin dependent kinase 4/6 (CDK4/6) inhibitor for the treatment of solid tumors and hematological malignancies. This invention also relates to the use of the combination thereof, in the management of hyperproliferative diseases like cancer.

### RELATED BACKGROUND ART

Tumor development is closely associated with genetic alteration and deregulation of CDKs and their regulators, suggesting that inhibitors of CDKs may be useful anti-cancer therapeutics. Indeed, early results suggest that transformed and normal cells differ in their requirement for, e.g., cyclin D/CDK4/6 and that it may be possible to develop novel antineoplastic agents devoid of the general host toxicity observed with conventional cytotoxic and cytostatic drugs.

The function of CDKs is to phosphorylate and thus activate or deactivate certain proteins, including e.g. retinoblastoma proteins, lamins, histone H1, and components of the mitotic spindle. The catalytic step mediated by CDKs involves a phospho-transfer reaction from ATP to the macromolecular enzyme substrate. Several groups of compounds (reviewed in e.g. Fischer, P. M. Curr. Opin. Drug Discovery Dev. 2001, 4, 623-634) have been found to possess anti-proliferative properties by virtue of CDK-specific ATP antagonism.

At a molecular level mediation of CDK/cyclin complex activity requires a series of stimulatory and inhibitory phosphorylation, or dephosphorylation, events. CDK phosphorylation is performed by a group of CDK activating kinases (CAKs) and/or kinases such as wee1, Myt1 and Mik1. Dephosphorylation is performed by phosphatases such as cdc25(a & c), pp2a, or KAP.

CDK/cyclin complex activity may be further regulated by two families of endogenous cellular proteinaceous inhibitors: the Kip/Cip family, or the INK family. The INK proteins specifically bind CDK4 and CDK6. p16^{ink4} (also known as MTS1) is a potential tumour suppressor gene that is mutated, or deleted, in a large number of primary cancers. The Kip/Cip family contains proteins such as p21^{Cip1, Waf1}, p27^{Kip1} and p57^{kip2}, where p21 is induced by p53 and is able to inactivate the CDK2/cyclin(E/A) complex. Atypically low levels of p27 expression have been observed in breast, colon and prostate cancers. Conversely over expression of cyclin E in solid tumours has been shown to correlate with poor patient prognosis. Over expression of cyclin D1 has been associated with oesophageal, breast, squamous, and non-small cell lung carcinomas.

The pivotal roles of CDKs, and their associated proteins, in co-ordinating and driving the cell cycle in proliferating cells have been outlined above. Some of the biochemical pathways in which CDKs play a key role have also been described. The development of monotherapies for the treatment of proliferative disorders, such as cancers, using therapeutics targeted generically at CDKs, or at specific CDKs, is therefore potentially highly desirable. Thus, there is a continued need to find new therapeutic agents to treat human diseases.

mTOR is a kinase protein predominantly found in the cytoplasm of the cell act, as a central regulator of many biological processes related to cell proliferation, angiogenesis, and cell metabolism. mTOR exerts its effects primarily by turning on and off the cell's translational machinery, which includes the ribosomes, and is responsible for protein synthesis. mTOR is a key intracellular point of convergence for a number of cellular signaling pathways. mTOR performs its regulatory function in response to activating or inhibitory signals transmitted through these pathways, which are located upstream from mTOR in the cell. These diverse signaling pathways are activated by a variety of growth factors (including vascular endothelial growth factors (VEGFs), platelet-derived growth factor (PDGF), epidermal growth factor (EGF), insulin-like growth factor 1 (IGF-1)), hormones (estrogen, progesterone), and the presence or absence of nutrients (glucose, amino acids) or oxygen. One or more of these signaling pathways may be abnormally activated in patients with many different types of cancer, resulting in deregulated cell proliferation, tumor angiogenesis, and abnormal cell metabolism.

### BRIEF SUMMARY OF THE INVENTION

The invention provides a combination comprising a first agent that inhibits the CDK4/6 pathway and a second agent that inhibits mTOR, ie the kinase activity of mTOR and its downstream effectors. In another aspect, the invention provides combinations including pharmaceutical compositions comprising a therapeutically effective amount of a first agent that inhibits CDK4/6, a second agent that inhibits the kinase activity of mTOR and downstream effectors, and a pharmaceutically acceptable carrier.

Furthermore, the present invention provides for the use of a therapeutically effective amount of a combination comprising a first agent that inhibits the CDK4/6 pathway and a second agent that inhibits the kinase activity of mTOR and downstream effectors, or a pharmaceutically acceptable salt or pharmaceutical composition thereof, in the manufacture of a medicament for treating cancer.

The present invention has a therapeutic use in the treatment of cancer, particularly retinoblastoma protein (retinoblastoma tumor suppressor protein or pRb) positive cancers. Types of such cancers include mantle cell lymphoma, pancreatic cancer, breast cancer, non small cell lung cancer, melanoma, colon cancer, esophageal cancer and liposarcoma.

The above combination and compositions can be administered to a system comprising cells or tissues, as well as a human patient or and animal subject.

### BRIEF DESCRIPTION OF THE DRAWINGS

FIG. 1 shows enhanced growth inhibitions by CDK4/6 and mTOR inhibitor combinations. Jeko-1 mantle cell lymphoma cells were used to evaluate the effects on cell growth. % growth compared to control (100%) is shown. Compound A1 is a CDK416 inhibitor and Compound B1 is an mTOR inhibitor. A1+B1 combinations are growth inhibitions observed when Jeko-1 cells were co-treated with A1 and B1 compounds at the same time. Actual concentrations used are shown in the graphs.

FIG. 2 is an isobologram analysis of a CDK4/6 and mTOR inhibitor combination in a Jeko-1 mantle cell lymphoma cell line. Compound A1 and B1 are CDK4/6 and mTOR inhibitors, respectively. The graph shown was constructed using the concentrations that gave 50% growth inhibitions. Dotted Line 1 represents the growth inhibitions predicted for a simple additivity when the effects of A1 and B1 are combined. Line 2 is the observed growth inhibitions, indicating that A1/B1 combination results in strong synergistic growth inhibition.

FIG. 3 is an isobologram analysis of a CDK4/6 and mTOR inhibitor combination in a MDA-MB453 breast cancer cell line. Compound A1 and B1 are CDK4/6 and mTOR inhibitors, respectively. Similar to FIG. 2 above, the graph shown was constructed using the concentrations that gave 50% growth inhibitions, with dotted Line 1 representing the growth inhibitions predicted for a simple additivity. Line 2 is the observed growth inhibitions, indicating that A1/B1 combination results in strong synergistic growth inhibition.

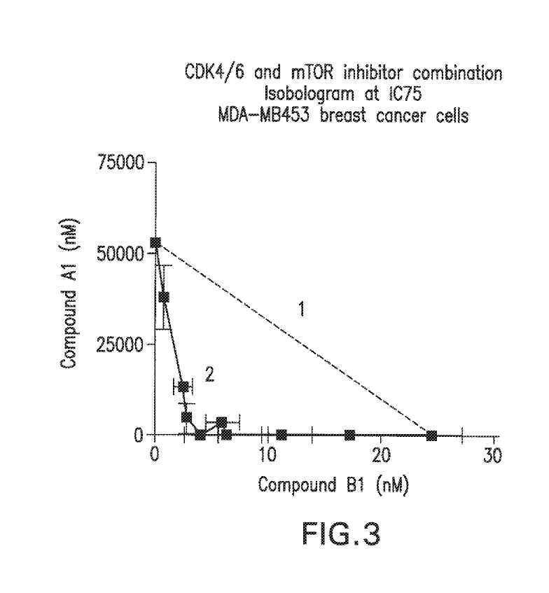

FIG. 4 shows that a combination of Compound A1 with Compound B1 enhanced tumor growth delay in the Jeko-1 mantle cell lymphoma xenograft model. Dosing was stopped 35 days post treatment initiation (56 days post implantation) and tumors were allowed to re-grow. The combination dosing group had significantly enhanced tumor growth delay (20 days).

FIGS. 5A and 5B illustrate a combination of Compound A1 with Compound B1 that enhanced tumor growth delay and tumor growth inhibition in the PANC-1 pancreatic carcinoma xenograft model, for tumor volume (FIG. 5A) and percentage alive (FIG. 5B). Dosing was stopped 22 days post treatment initiation and tumors were allowed to re-grow. The combination dosing group had significantly enhanced tumor growth delay (18 days).

FIGS. 6A, 6B, and 6C illustrate, when the combination of CDK4/6 inhibitor Compound A1 and mTOR inhibitor Compound B1, is used to treat Jeko-1 cells, the resulting inhibition values were used by CHALICE software to generate Inhibition and ADD Excess Inhibition matrices, as well as the isobolograms.

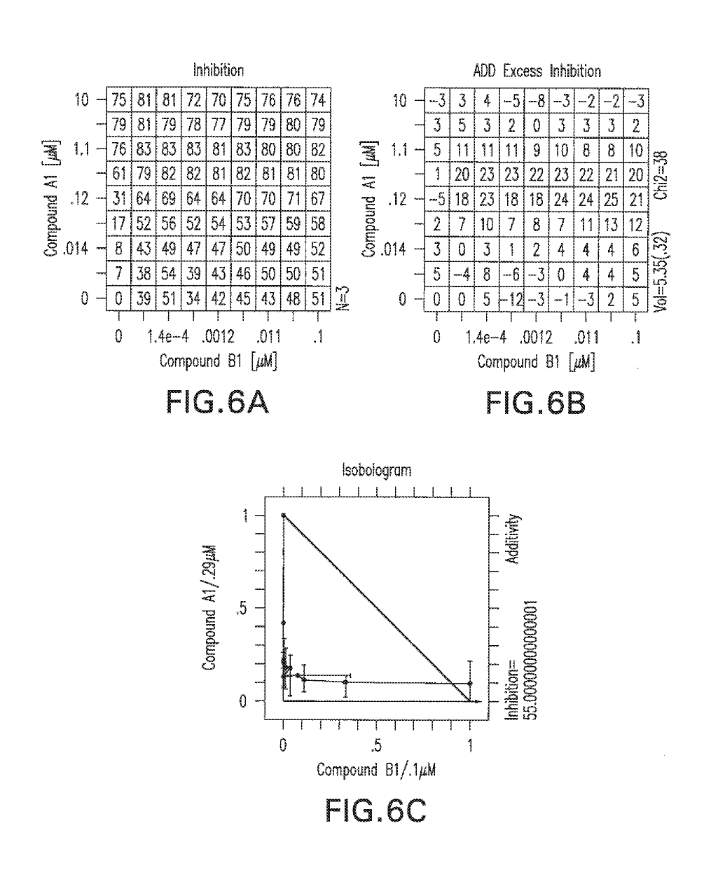

FIGS. 7A, 7B, and 7C illustrate, when the combination of CDK4/6 inhibitor Compound A1 and mTOR inhibitor Compound B2, is used to treat Jeko-1 cells, the resulting inhibition values were used by CHALICE software to generate Inhibition and ADD Excess Inhibition matrices, as well as the isobolograms.

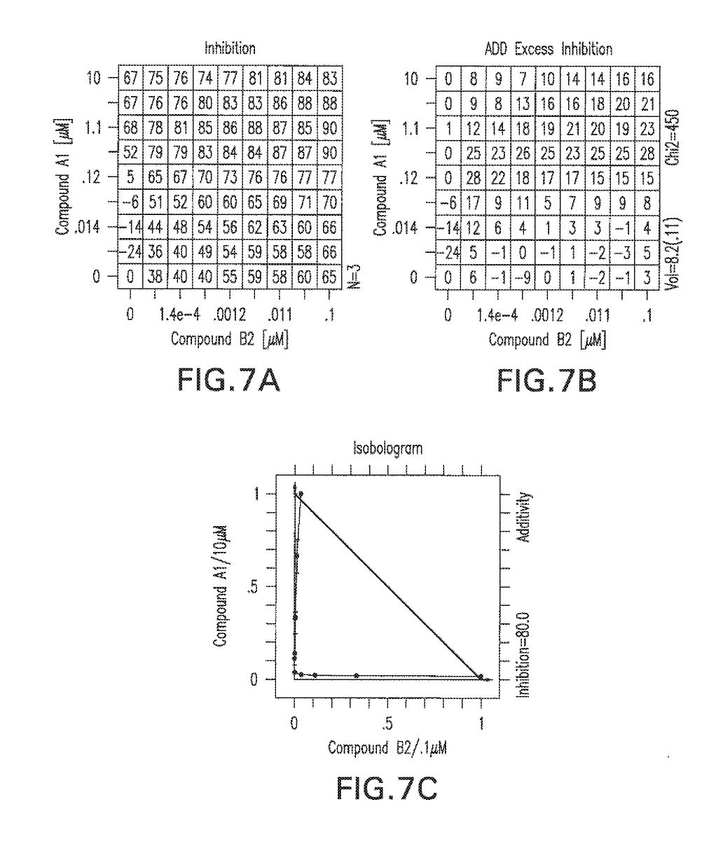

FIGS. 8A, 8B, and 8C illustrate, when the combination of CDK4/6 inhibitor Compound A4 and mTOR inhibitor Compound B1, is used to treat Jeko-1 cells, the resulting inhibition values were used by CHALICE software to generate Inhibition and ADD Excess Inhibition matrices, as well as the isobolograms.

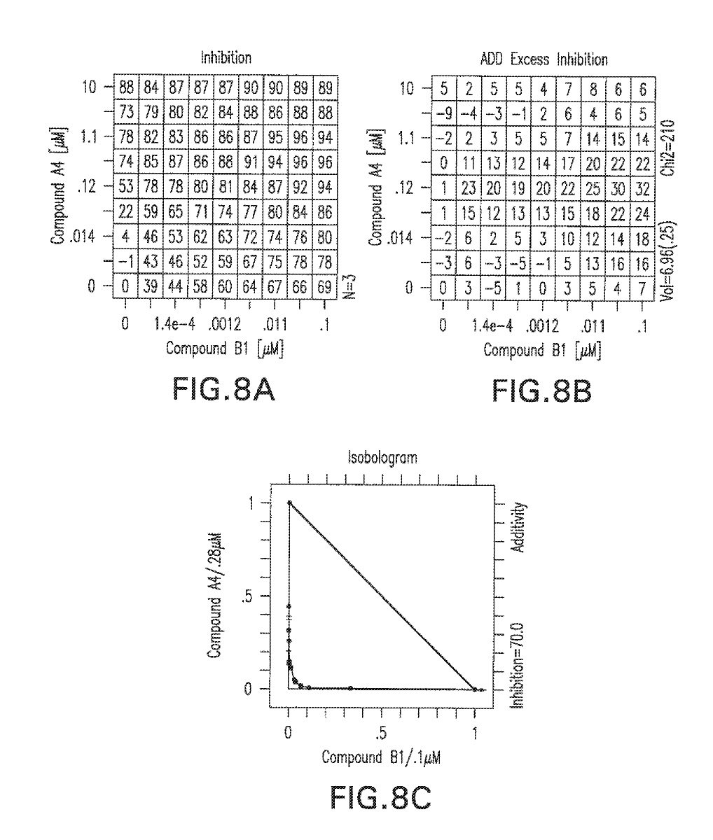

FIGS. 9A, 9B, and 9C illustrate, when the combination of CDK4/6 inhibitor Compound A2 and mTOR inhibitor Compound B1, is used to treat Jeko-1 cells, the resulting inhibition values were used by CHALICE software to generate Inhibition and ADD Excess Inhibition matrices, as well as the isobolograms.

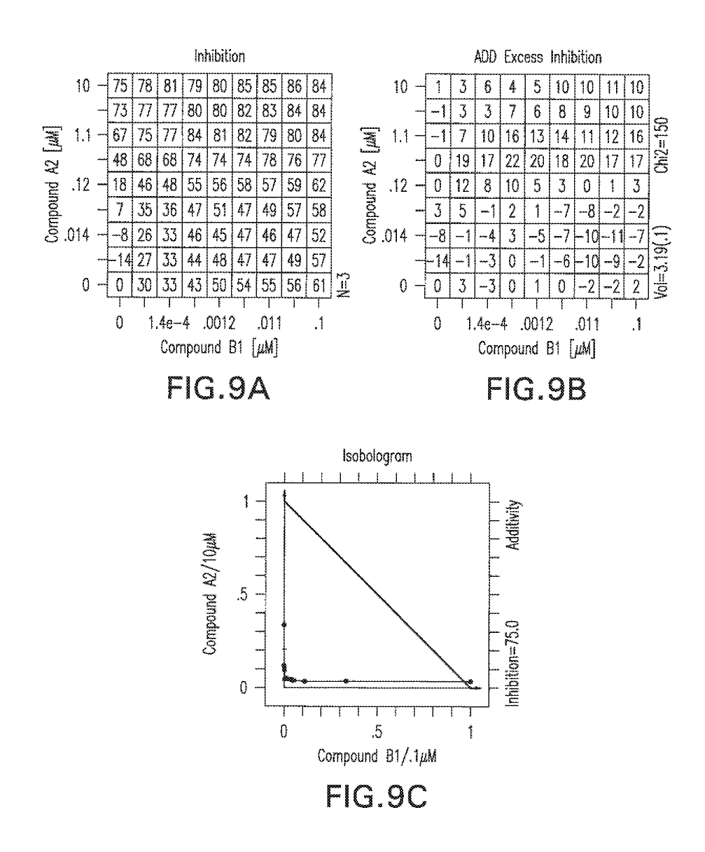

FIGS. 10A, 10B, and 10C illustrate, when the combination of CDI 4/6 inhibitor Compound A3 and mTOR inhibitor Compound B1, is used to treat Jeko-1 cells, the resulting inhibition values were used by CHALICE software to generate Inhibition and ADD Excess Inhibition matrices, as well as the isobolograms.

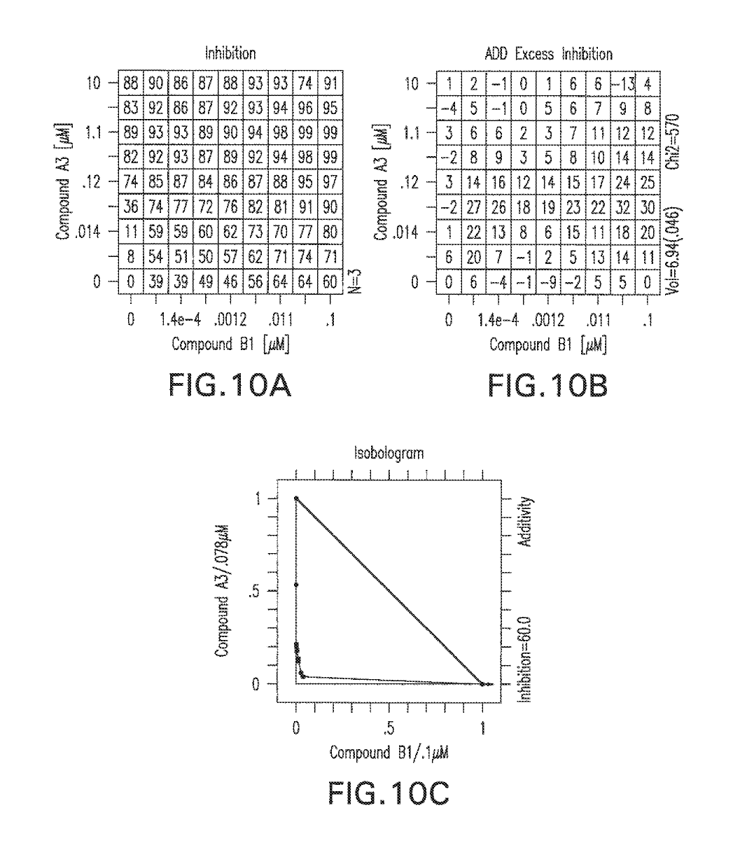

FIGS. 11A, 11B, and 11C illustrate, when the combination of CDK4/6 inhibitor Compound A6 and mTOR inhibitor Compound B1, is used to treat Jeko-1 cells, the resulting inhibition values were used by CHALICE software to generate Inhibition and ADD Excess Inhibition matrices, as well as the isobolograms.

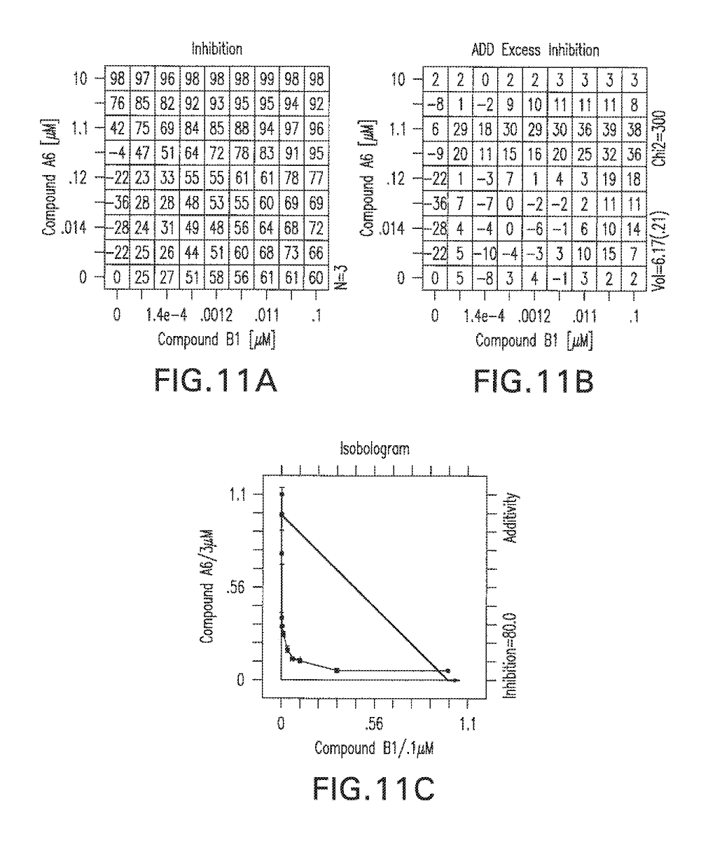

FIGS. 12A, 12B, and 12C illustrate, when the combination of CDK4/6 inhibitor Compound A5 and mTOR inhibitor Compound 1B, is used to treat Jeko-1 cells, the resulting inhibition values were used by CHALICE software to generate Inhibition and ADD Excess Inhibition matrices, as well as the isobolograms.

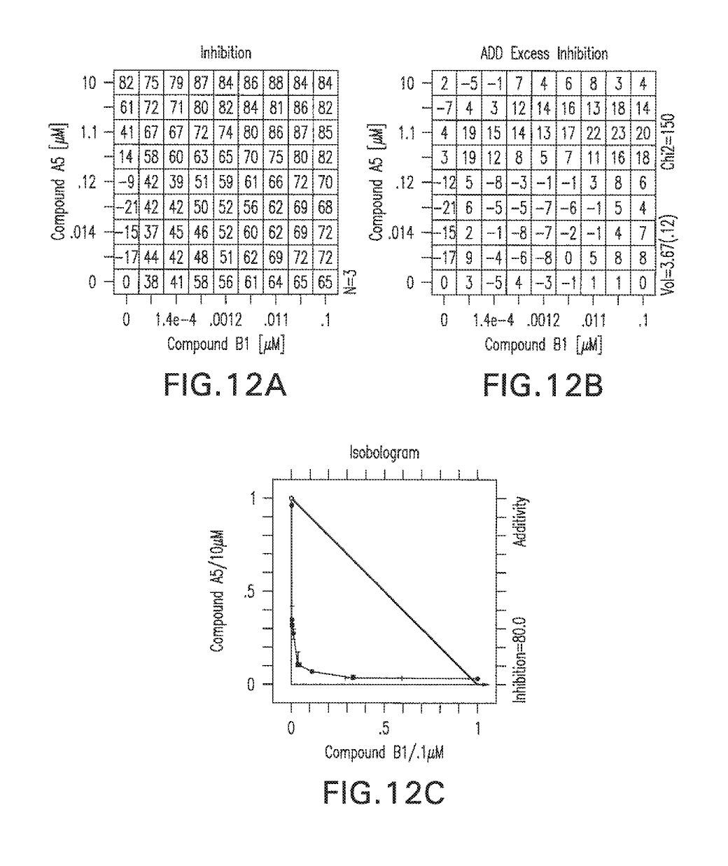

FIGS. 13A, 13B, and 13C illustrate, when the combination of CDK4/6 inhibitor Compound A4 and mTOR inhibitor Compound B2, is used to treat Jeko-1 cells, the resulting inhibition values were used by CHALICE software to generate inhibition and ADD Excess Inhibition matrices, as well as the isobolograms.

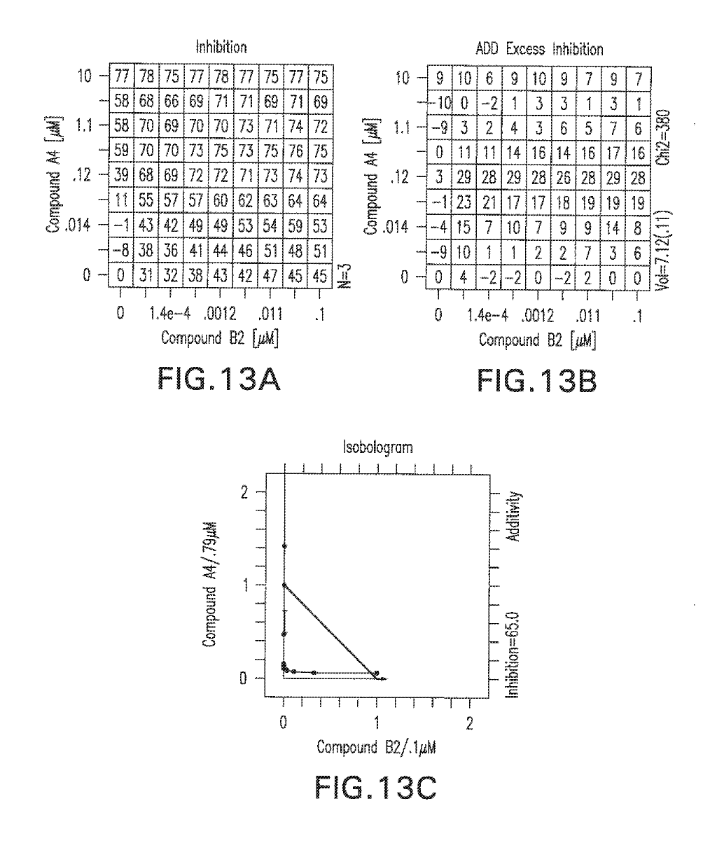

FIGS. 14A, 14B, and 14C illustrate, when the combination of CDK4/6 inhibitor Compound A2 and mTOR inhibitor Compound B2, is used to treat Jeko-1 cells, the resulting inhibition values were used by CHALICE software to generate Inhibition and ADD Excess Inhibition matrices, as well as the isobolograms.

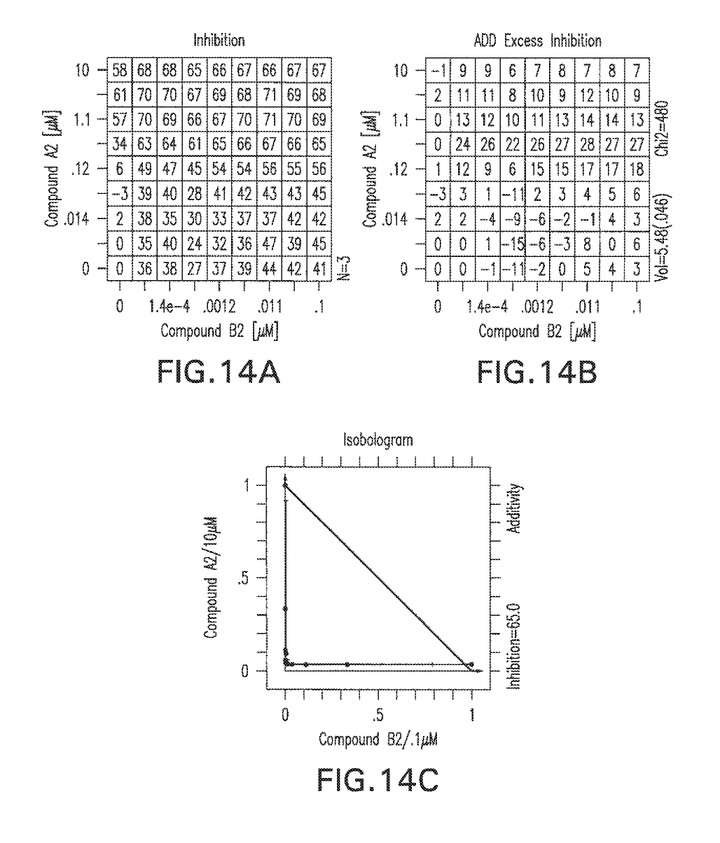

FIGS. 15A, 15B, and 15C illustrate, when the combination of CDK4/6 inhibitor Compound A3 and mTOR inhibitor Compound B2, is used to treat Jeko-1 cells, the resulting inhibition values were used by CHALICE software to generate Inhibition and ADD Excess Inhibition matrices, as well as the isobolograms.

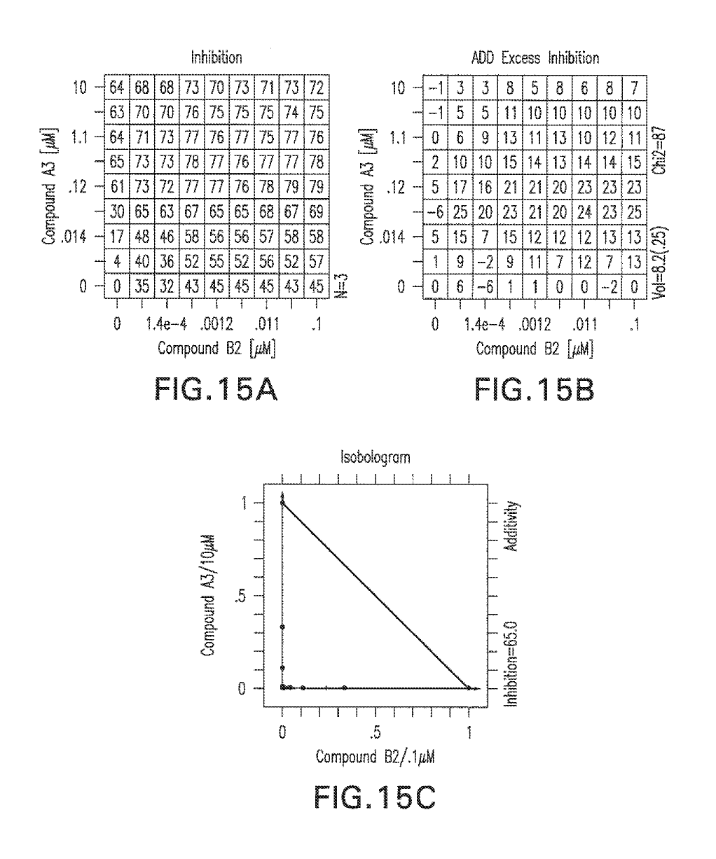

FIGS. 16A, 16B, and 16C illustrate, when the combination of CDK4/6 inhibitor Compound A6 and mTOR inhibitor Compound B2, is used to treat Jeko-1 cells, the resulting inhibition values were used by CHALICE software to generate Inhibition and ADD Excess Inhibition matrices, as well as the isobolograms.

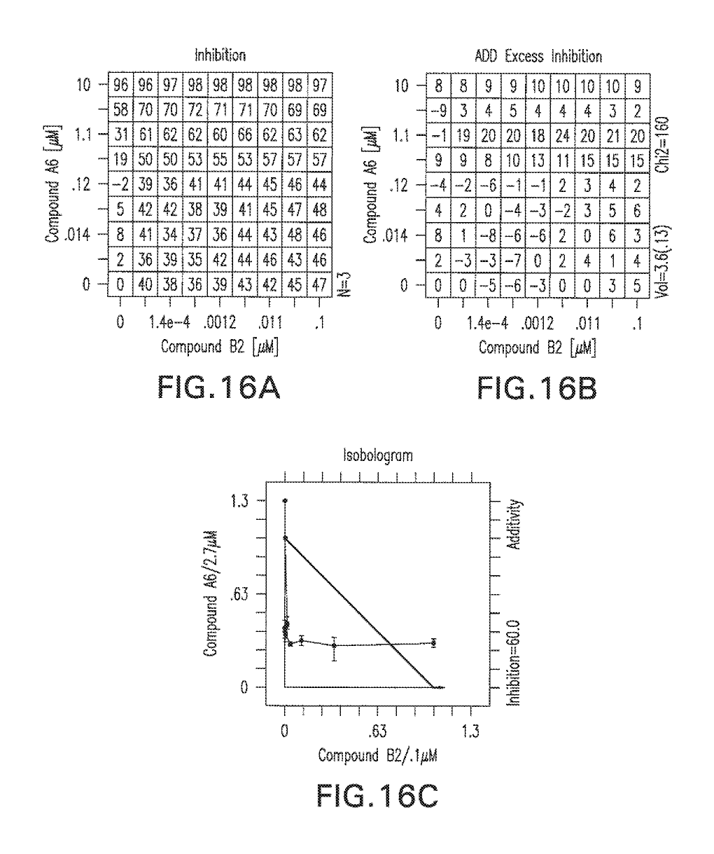

FIGS. 17A, 17B, and 17C illustrate, when the combination of CDK4/6 inhibitor Compound A5 and mTOR inhibitor Compound B2, is used to treat Jeko-1 cells, the resulting inhibition values were used by CHALICE software to generate Inhibition and ADD Excess Inhibition matrices, as well as the isobolograms.

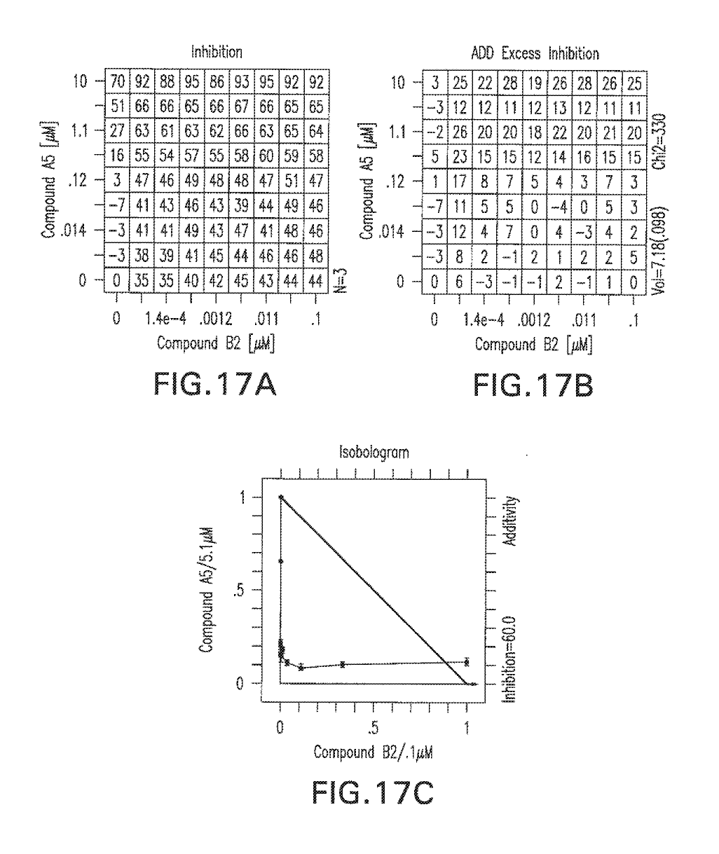

### DETAILED DESCRIPTION OF THE INVENTION

Mammalian cell cycle progression is a tightly controlled process in which transitions through different phases are conducted in a highly ordered manner and guarded by multiple checkpoints. The retinoblastoma protein (pRb) is the checkpoint protein for G1 to S phase transition, which associates with a family of E2F transcription factors to prevent their activity in the absence of appropriate growth stimuli. Upon mitogen stimulation, quiescent cells begin their entry into S phase by newly synthesizing D-cyclins, which are the activators of cyclin dependent kinases 4 and 6 (CDK4/6). Once bound by the cyclins, CDK4/6 deactivate the pRb protein via phosphorylation and this releases E2F to direct transcription of genes required for S phase. Full deactivation of pRb requires phosphorylations by both cyclin D-CDK4/6 and cyclin E-CDK2, where phosphorylations by CDK4/6 at specific sites of pRb (Ser780, Ser795) have been shown to be a prerequisite for cyclin E-CDK2 phosphorylation. In addition to D-cyclins, the activity of CDK4/6 is regulated by p16, encoded by INK4a gene, which inhibits the kinase activity. The CIP/KIP proteins, which are the inhibitors of cyclin E-CDK2, also bind to cyclin D-CDK4/6 complex, and this results in further activation of CDK2 by sequestering the CIP/KIP away from their target. Therefore, the cyclin D-CDK4/6 is a key enzyme complex that regulates the G1 to S phase transition.

The D-cyclin-CDK4/6-INK4a-pRb pathway is universally disrupted to favor cell proliferation in cancer. In a majority of cases (˜80%), cancers maintain a functional pith and utilize different mechanisms to increase the CDK4/6 kinase activity. One of the most common events is the inactivation of p16 via mutations, deletions and epigenetic silencing. Indeed, the functional absence of p16 is frequently observed in large portions of non small cell lung cancer, melanoma, pancreatic cancer and mesothelioma Coupled with the observation that a specific mutation of the CDK4 gene (CDKR24C), that confers resistance to p16 binding, has been shown to play a causal role in a familial melanoma, the growth advantage provided by unchecked CDK4/6 activity appear to be one of the key elements associated with a tumor development.

Another mechanism to enhance the kinase activity is to increase the abundance of D-cyclins and this is accomplished by translocation, amplification and overexpression of the gene. Cyclin D1 gene is translocated to the immunoglobulin heavy chain in a majority of mantle cell lymphoma and this aberration leads to constitutive expression of the gene resulting in unchecked cell proliferation. The translocation is also observed in many cases of multiple myeloma. The example of the gene amplification is seen in squamous cell esophageal cancer, where approximately 50% of the cases have been reported to harbor cyclin D1 amplifications. This suggests that a large portion of the esophageal cancer may be highly dependent on activated kinases for growth. Cyclin D1amplification is also often detected in breast cancers. In addition to the genetic defects directed related to the cyclin D1 gene, its transcription can also be profoundly elevated by activated oncogenes that are upstream regulators of the gene. Activated Ras or Neu oncogenes have been shown to promote breast cancer in mice by primarily upregulating cyclin D1. Suppression of the cyclin D1 levels or inhibition of the kinase activity were able to prevent tumor growth in both initiation and maintenance phases, demonstrating that an unchecked CDK4/6 was the key element in the development of the cancers. Other activating aberrations of mitogen pathways such as V600E B-Raf in MAPK and PTEN deletions in PI3K also increase D-cyclins to achieve accelerated proliferations, suggesting CDK4/6 may also be crucial for the cancers bearing the. Lastly, the genes encoding CDK4 and 6 are also amplified in subset of human neoplasms. CDK4 gene is amplified in 100% of liposarcomas along with MDM2 gene, while CDK6 is frequently amplified in T-LBL/ALL. Taken together, CDK4/6 appears to be a crucial protein necessary for proliferation of numerous human cancers with a functional pRb, including mantle cell lymphoma, pancreatic cancer, breast cancer, non small cell Jung cancer, melanoma, colon cancer, esophageal cancer and liposarcoma.

### First General Embodiment of the Invention

A combination comprising a first agent that is a cyclin dependent kinase 416(CDK4/6) inhibitor and a second agent that is an mTOR inhibitor, wherein the first agent is a compound of Formula I:

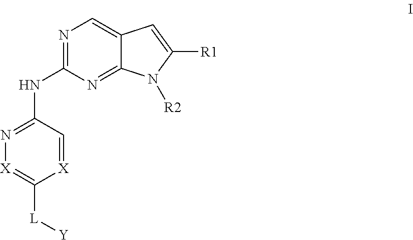

or a pharmaceutically acceptable salt thereof, wherein

X is CR⁹, or N;

R¹ is C₁₋₈alkyl, CN, C(O)OR⁴ or CONR⁵R⁶, a 5-14 membered heteroaryl group, or a 3-14 membered cycloheteroalkyl group;

R² is C₁₋₈alkyl, C₃₋₁₄cycloalkyl, or a 5-14 membered heteroaryl group, and wherein le may be substituted with one or more C₁₋₈alkyl, or OH;

L is a bond, C₁₋₈alkylene, C(O), or C(O)NR¹⁰, and wherein L may be substituted or unsubstituted;

Y is H, R¹¹, NR¹²R¹³, OH, or Y is part of the following group

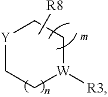

where Y is CR⁹ or N;

where 0-3 R⁸ may be present, and R⁸ is C₁₋₈alkyl oxo, halogen, or two or more R⁸ may form a bridged alkyl group;

W is CR⁹, or N;

R³ is H, C₁₋₈cycloalkyl, C₁₋₈alkylR¹⁴, C₃₋₁₄cycloalkyl, C(O)C₁₋₈ alkyl, C₁₋₈haloalkyl, C₁₋₈alkylOH, C(O)NR¹⁴R¹⁵, C₁₋₈cyanoalkyl, C(O)R¹⁴, C₀₋₈alkylC(O)C₀₋₈alkylNR¹⁴R¹⁵, C₀₋₈alkylC(O)OR¹⁴, NR¹⁴R¹⁵, SOO₂C₁₋₈alkyl, C₁₋₈alkylC₃₋₁₄cycloalkyl, C(O)C₁₋₈alkylC₃₋₁₄cycloalkyl, C₁₋₈alkoxy, or OH which may he substituted or unsubstituted when R³ is not H.

R⁹ is H or halogen;

R⁴, R⁵, R⁶, R⁷, R¹⁰, R¹¹, R¹², R¹³, R¹⁴, and R¹⁵ are each independently selected from H, C₁₋₈alkyl, C₃₋₁₄ cycloalkyl, a 3-14 membered cycloheteroalkyl group, a C₆₋₁₄ aryl group, a 5-14 membered heteroaryl group, alkoxy, C(O)H, C(N)OH, C(N)OCH₃, C(O)C₁₋₃alkyl, C₁₋₈alkylNH₂, C₁₋₆alkylOH, and wherein R⁴, R⁵, R⁶, R⁷, R¹⁰, R¹¹, R¹², and R¹³, R¹⁴, and R¹⁵ when not H may be substituted or unsubstituted;

m and n are independently 0-2; and

wherein L, R³, R⁴, R⁵, R⁶, R⁷, R¹⁰, R¹¹, R¹², and R¹³, R¹⁴, and R¹⁵ may be substituted with one or more of C₁₋₈alkyl, C₂₋₈alkynyl, C₃₋₁₄cycloalkyl, 5-14 membered heteroaryl group, C₆₋₁₄aryl group, a 3-14 membered cycloheteroalkyl group, OH, (O), CN, alkoxy, halogen, or NH₂.

In an embodiment of the first general embodiment, the combination includes a CDK4/6 inhibitor of Formula I, wherein R³ is H, C₃₋₁₄cycloalkyl, C(O)C₁₋₈ alkyl, C₁₋₈alkylOH, C₁₋₈cyanoalkyl, C₀₋₈alkylC(O)C₀₋₈alkylNR¹⁴R¹⁵, C₀₋₈alkylC(O)OR¹⁴, NR¹⁴R¹⁵, C₁₋₈alkylC₃₋₁₄cycloalkyl, C(O)C₁₋₈alkylC₃₋₁₄cycloalkyl, C₀₋₈alkoxy, C₁₋₈alkylR¹⁴, C₁₋₈haloalkyl, or C(O)R¹⁴, which may be substituted with one or more of OH, CN, F, or NH₂, and wherein R¹⁴ and R¹⁵ are each independently selected from H, C₁₋₈ alkyl, alkoxy, C(O)C₁₋₃alkyl, C₁₋₃alkylNH₂, or C₁₋₆alkylOH.

In another embodiment of the first general embodiment, the combination includes a CDK4/6 inhibitor of Formula I, wherein R³ is H, C₁₋₈alkyl, or C₁₋₈alkylOH. In yet another embodiment, the inventive combination includes a CDK4/6 inhibitor or Formula I, where Y is H, OH, or Y is part of the following group

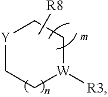

where Y is N and W is CR⁹, or N; and where 0-2 R⁸ may be present, and R⁸ is C₁₋₈alkyl, oxo, or two or more R⁸ may form a bridged alkyl group.

In yet another embodiment of the first general embodiment, the present invention includes a CDK4/6 inhibitor of Formula I where L is a bond, C₁₋₈alkylene, or C(O)NH, or C(O). In another preferred embodiment, the combination includes a CDK4/6 inhibitor of Formula I, Where R² is C₃₋₁₄cycloalkyl. In another embodiment, R² is cyclopentane.

In yet another embodiment of the first general embodiment, the present invention includes a CDK4/6 inhibitor of Formula I where R¹ is CN, C(O)OR⁴, CONR⁵R⁶, or a 5-14 membered heteroaryl group. In yet another embodiment, R¹ is CONR⁵R⁶, and R⁵ and R⁶ are C₁₋₈alkyl.

In yet another embodiment, the present invention includes a CDK4/6 inhibitor of Formula I where X is CR⁹. In another embodiment, one X is N and the other X is CR⁹. In another embodiment, the combination includes CDK4/6 inhibitor of Formula I, where X is CR⁹ and Y is

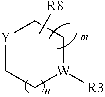

where m and n are 1, and Y and W are N.

In another embodiment of the first general embodiment, the present invention includes CDK4/6 inhibitors of Formula I wherein one X is N and the other X is CR⁹. In an embodiment, the present invention includes compounds of Formula (I), such as:

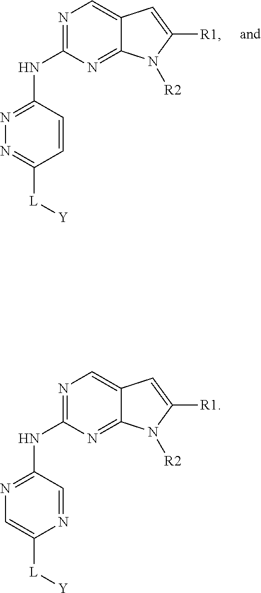

In another embodiment of the first general embodiment, the present invention includes compounds of Formula I wherein X is CR⁹ and Y is

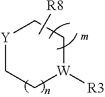

where m and n are 1, and Y and W are N.

In another embodiment of Formula I, R³ is H, C₁₋₈alkyl, C₃₋₁₄cycloalkyl, C(O)C₁₋₈ alkyl, C₁₋₈alkylOH, C₁₋₈cyanoalkyl, C₀₋₈alkylC(O)C₀₋₈alkylNR¹⁴R¹⁵, C₀₋₈alkylC(O)OR¹⁴, NR¹⁴R¹⁵, C₁₋₈alkylC₃₋₁₄cycloalkyl, C(O)C₁₋₈alkylC₃₋₁₄cycloalkyl, C₀₋₈alkoxy, C₁₋₈ alkylR¹⁴, C₁₋₈haloalkyl, or C(O)R¹⁴, which may be substituted with one or more of OH, CN, F, or NH₂, and wherein R¹⁴ and R¹⁵ are each independently selected from H, alkyl, C₃₋₁₄cycloalkyl, alkoxy, C(O)C₁₋₃alkyl, C₁₋₈alkylNH₂, or C₁₋₆alkylOH.

In another embodiment of Formula I, Y is OH, or Y is part of the following group

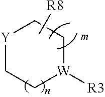

where Y is N and W is CR⁹, or N;

where 0-2 R⁸ may be present, and R⁸ is C₁₋₈alkyl, oxo, or two or more R⁸ may form a bridged alkyl group.

In another embodiment of Formula I,

L is a bond, C₁₋₈alkylene, or C(O)NH, or C(O).

R² is any one of a C₃₋₇cycloalkyl.

R¹ is CN, C(O)OR⁴, CONR⁵R⁶, or a 5-14 membered heteroaryl group.

In another embodiment Formula I, X is CR⁹ or X is N and the other X is CR⁹ or X is CR⁹ and Y is

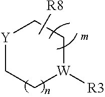

where m and n are I, and Y and W are N.

Preferred compounds of Formula I include:

7-Cyclopentyl-2-[5-(3-methyl-piperazin-yl)-pyridin-2-ylamino]-7H-pyrrolo[2,3d]pyrimidine-6-carbonitrile;

7-Cyclopentyl-2-{5-[4-(2-fluoro-ethyl)-piperazin-1-yl]-pyridin-2-ylamino}-7H-pyrrolo[2,3-d]pyrimidine-6-carboxylic acid dimethylamide;

7-Cyclopentyl-2-(4-dimethylamino-3,

4,5,6-tetrahydro-2H-[1,3′]bipyridinyl-6′-ylamino)-7H-pyrrolo[2,3-d]pyrimidine-6-carboxylic acid dimethylamide;

2-[5-(4-Carbamoylmethyl-piperazin-1-yl)-pyridin-2-ylamino]-7-cyclopentyl-7H-pyrrolo[2,3-d]pyrimidine-6-carboxylic acid dimethylamide;

2-{5-[4-(2-Amino-acetyl)-piperazin-1-yl]-pyridin-2-ylamino}-7-cyclopentyl-7H-pyrrolo[2,3-d]pyrimidine-6-carboxylic acid dimethylamide;

2-[5-(3-Amino-pyrrolidin-1-yl)-pyridin-2-ylamino]-7-cyclopentyl-7H-pyrrolo[2,3-d]pyrimidine-6-carboxylic acid dimethylamide;

7-Cyclopentyl-2-{5-[4-(2-methoxy-ethyl)-piperazin-1-yl]-pyridin-2-ylamino}-7H-pyrrolo[2,3-d]pyrimidine-6-carboxylic acid dimethylamide;

7-Cyclopentyl-2-[4-(2-hydroxyethyl)-3,4,5,6-tetrahydro-2H-[1,2′]bipyrazinyl-5′-ylamino]-7H-pyrrolo[2,3-d]pyrimidine-6-carboxylic acid dimethylamide;

7-Cyclopentyl-2-[5-((R)-3-methyl-piperazin-1-yl)-pyridin-2-ylamino]-7H-pyrrolo[2,3-d]pyrimidine-6-carboxylic acid dimethylamide;

7-Cyclopentyl-2-[5-((S)-3-methylpiperazin-1-yl)-pyridin-2-ylamino]-7H-pyrrolo[2,3-d]pyrimidine-6-carboxylic acid dimethylamide;

7-Cyclopentyl-2-[5-(3-methylpiperazin-1-yl)-pyridin-2-ylamino]-7H-pyrrolo[2,3-d]pyrimidine-6-carboxylic acid dimethylamide;

7-Cyclopentyl-2-{5-[4-(3-hydroxypropyl)-piperazin-1-yl]-pyridin-2-ylamino}-7H-pyrrolo[2,3-d]pyrimidine-6-carboxylic acid dimethylamide;

7-Cyclopentyl-2-{5-[4-(pyrrolidine-1-carbonyl)-piperazin-1-yl]-pyridin-2-ylamino}-7H-pyrrolo[2,3-d]pyrimidine-6-carboxylic acid dimethylamide;

7-Cyclopentyl-2-{5-[4-(2-hydroxy-ethyl)-piperazin-1-yl]-pyridin-2-ylamino}-7H-pyrrolo[2,3-d]pyrimidine-6-carboxylic acid dimethylamide;

7-Cyclopentyl-2-{5-[4-((S)-2,3-dihydroxypropyl)-piperazin-1-yl]-pyridin-2-ylamino}-7H-pyrrolo[2,3-d]pyrimidine-6-carboxylic acid dimethylamide;

7-Cyclopentyl-2-(5-{4-[2-(2-hydroxyethoxy)-ethyl]-piperazin-1-yl) -pyridin-2-ylamino}-7H-pyrrolo[2,3-d]pyrimidine-6-carboxylic acid dimethylamide;

7-Cyclopentyl-2-{5-[4-(2-hydroxy-1-methylethyl)-piperazin-1-yl]-pyridin-2-ylamino}-7H-pyrrolo[2,3-d]pyrimidine-6-carboxylic acid dimethylamide;

7-Cyclopentyl-2-{6-[4-(2-hydroxyethyl)-piperazin-1-yl]-pyridazin-3-ylamino}-7H-pyrrolo[2,3-d]pyrimidine-6-carboxylic acid dimethylamide;

7-Cyclopentyl-2-{5-[4-(2,3-dihydroxypropyl)-piperazin-1-yl]-pyridin-2-ylamino}-7H-pyrrolo[2,3-d]pyrimidine-6-carboxylic acid dimethylamide;

7-Cyclopentyl-2-{5-[4-((R)-2,3-dihydroxypropyl)-piperazin-1-yl]-pyridin-2-ylamino}-7H-pyrrolo[2,3-d]pyrimidine-6-carboxylic acid dimethylamide;

7-Cyclopentyl-2-(4-dimethylamino-3,4,5,6-tetrahydro-2H-[1,3′]bipyridinyl-6′-ylamino)-7H-pyrrolo[2,3-d]pyrimidine-6-carbonitrile;

7-Cyclopentyl-2-(3,4,5,6-tetrahydro-2H-[1,2′]bipyrazinyl-5′-ylamino)-7H-pyrrolo[2,3-d]pyrimidine-6-carboxylic acid dimethylamide;

7-Cyclopentyl-2-[5-(piperazine-1-carbonyl)-pyridin-2-ylamino]-7H-pyrrolo[2,3d]pyrimidine-6-carboxylic acid dimethylamide;

7-Cyclopentyl-2-[5-(4-dimethylaminopiperidine-1-carbonyl)-pyridin-2-ylamino]-7H-pyrrolo[2,3-d]pyrimidine-6-carboxylic acid dimethylamide;

7-Cyclopentyl-2-(1′,2′,3′,4′,5′,6′-hexahydro-[3,4′]bipyridinyl-6-ylamino)-7H-pyrrolo[2,3d]pyrimidine-6-carboxylic acid dimethylamide;

7-Cyclopentyl-2-[5-((S)-3-methylpiperazin-1-ylmethyl)-pyridin-2-ylamino]-7Hpyrrolo[2,3-d]pyrimidine-6-carboxylic acid dimethylamide;

7-Cyclopentyl-2-{5-[4-((S)-2-hydroxypropyl)-piperazin-1-yl]-pyridin-2-ylamino}-7H-pyrrolo[2,3-d]pyrimidine-6-carboxylic acid dimethylamide;

7-Cyclopentyl-2-{5-[4-((R)-2-hydroxypropyl)-piperazin-1-yl]-pyridin-2-ylamino}-7H-pyrrolo[2,3-d]pyrimidine-6-carboxylic acid dimethylamide;

7-Cyclopentyl-2-(5-piperazin-1-yl-pyridin-2-ylamino)-7H-pyrrolo[2,3-d]pyrimidine-6-carboxylic acid methylamide;

7-Cyclopentyl-2-[(5-(4-isopropyl-piperazin-1-yl)-pyridin-2-ylamino]-7H-pyrrolo[2,3d]pyrimidine-6-carboxylic acid dimethylamide;

7-Cyclopentyl-2-[5-(4-isopropyl-piperazine-1-carbonyl)-pyridin-2-ylamino]-7H-pyrrolo[2,3-d]pyrimidine-6-carboxylic acid dimethylamide;

7-Cyclopentyl-2-{5-[4-(4-methyl-pentyl)-piperazin-1-yl]-pyridin-2-ylamino}-7H-pyrrolo[2,3-d]pyrimidine-6-carboxylic acid dimethylamide;

7-Cyclopentyl-2-[6-(4-isopropyl-piperazin-1-yl)-pyridazin-3-ylamino]-7H-pyrrolo[2,3-d]pyrimidine-6-carboxylic acid dimethylamide;

7-Cyclopentyl-2-{5-[4-(2-hydroxy-2methylpropyl)-piperazin-1-yl]-pyridin-2-ylamino}-7H-pyrrolo[2,3-d]pyrimidine-6-carboxylic acid dimethylamide;

7-Cyclopentyl-2-[5-(3,3-dimethyl-piperazin-1-yl)-pyridin-2-ylamino]-7H-pyrrolo[2,3-d]pyrimidine-6-carboxylic acid dimethylamide;

7-Cyclopentyl-2-[5-(3,8-diaza-bicyclo[3.2.1]oct-3-ylmethyl)-pyridin-2-ylamino]-7H-pyrrolo[2,3-d]pyrimidine-6-carboxylic acid dimethylamide;

7-Cyclopentyl-2-(5-piperazin-1-yl-pyridin-2-ylamino)-7H-pyrrolo[2,3-d]pyrimidine-6-carboxylic acid dimethylamide;

7-Cyclopentyl-2-[5-(4-ethyl-piperazin-1-yl)-pyridin-2-ylamino]-7H-pyrrolo[2,3-d]pyrimidine-6-carboxylic acid dimethylamide;

7-Cyclopentyl-2-[5-(4-cyclopentyl-piperazin-1-yl)-pyridin-2-ylamino]-7H-pyrrolo[2,3-d]pyrimidine-6-carboxylic acid dimethylamide;

7-Cyclopentyl-2-(1′-isopropyl-1′,2′,3′,4′,5′,6′-hexahydro-[3,4′]bipyridinyl-6-ylamino)-7H-pyrrolo[2,3-d]pyrimidine-6-carboxylic acid dimethylamide;

7-Cyclopentyl-2-{5-[(R)-4-(2-hydroxyethyl)-3-methyl-piperazin-1-yl]-pyridin-2-ylamino}-7H-pyrrolo[2,3-d]pyrimidine-6-carboxylic acid dimethylamide;

7-Cyclopentyl-2-{5-[(S)-4-(2-hydroxyethyl)-3-methyl-piperazin-1-yl]-pyridin-2-ylamino}-7H-pyrrolo[2,3-d]pyrimidine-6-carboxylic acid dimethylamide;

7-Cyclopentyl-2-{5-[4-(2-hydroxyethyl)-piperazin-1-ylmethyl]-pyridin-2-ylamino}-7H-pyrrolo[2,3-d]pyrimidine-6-carboxylic acid dimethylamide;

7-Cyclopentyl-2-{5-[4-(2-dimethylaminoacetyl)-piperazin-1-yl]-pyridin-2-ylamino}-7H-pyrrolo[2,3-d]pyrimidine-6-carboxylic acid dimethylamide;

7-Cyclopentyl-2-{5-[4-(2-ethyl-butyl)piperazin-1-yl]-pyridin-2-ylamino}-7H-pyrrolo[2,3-d]pyrimidine-6-carboxylic acid dimethylamide;

2-{5-[4-(2-Cyclohexyl-acetyl)piperazin-1-yl]-pyridin-2-ylamino}-7-cyclopentyl-7H-pyrrolo[2,3-d]pyrimidine-6-carboxylic acid dimethylamide;

7-Cyclopentyl-2-{5-[4-(3-cyclopentyl-propionyl)-piperazin-1-yl]-pyridin-2-ylamino}7H-pyrrolo[2,3-d]pyrimidine-6-carboxylic acid dimethylamide;

7-Cyclopentyl-2-[5-(4-isobutylpiperazin-1-yl)-pyridin-2-ylamino]-7H-pyrrolo[2,3d]pyrimidine-6-carboxylic acid dimethylamide;

{4-[6-(7-Cyclopentyl-6-dimethylcarbamoyl-7H-pyrrolo[2,3-d]pyrimidin-2-ylamino)pyridin-3-yl]-piperazin-1-yl}-acetic acid methyl ester;

7-Cyclopentyl-2-{5-[4-(2-isopropoxyethyl)-piperazin-1-yl]-pyridin-2-ylamino}-7Hpyrrolo[2,3-d]pyrimidine-6-carboxylic acid dimethylamide;

{4-[6-(7-Cyclopentyl-6-dimethylcarbamoyl-7H-pyrrolo[2,3-d]pyrimidin-2-ylamino)pyridin-3-yl]-piperazin-1-yl}-acetic acid ethyl ester;

4-(6-{7-Cyclopentyl-6-[(2-hydroxy-ethyl)methyl-carbamoyl]-7H-pyrrolo[2,3-d]pyrimidin-2-ylamino}-pyridin-3-yl)piperazine-1-carboxylic acid tert-butyl ester;

7-Cyclopentyl-2-{5-[4-(2-methyl-butyl)piperazin-1-yl]-pyridin-2-ylamino}-7H-pyrrolo[2,3-d]pyrimidine-6-carboxylic acid dimethylamide;

7-Cyclopentyl-2-[1′-(2-hydroxy-ethyl)-1′,2′,3′,4′,5′,6′-hexahydro-[3,4′]bipyridinyl-6-ylamino]-7H-pyrrolo[2,3-d]pyrimidine-6-carboxylic acid dimethylamide;

{4-[6-(7-Cyclopentyl-6-dimethylcarbamoyl-7H-pyrrolo[2,3-d]pyrimidin-2-ylamino)-pyridin-3-yl]piperazin-1-yl}-acetic acid; and

2-{4-[6-(7-Cyclopentyl-6-dimethylcarbamoyl-7H-pyrrolo[2,3-d]pyrimidin-2-ylamino)-pyridin-3-yl]-piperazin-1-yl}-propionic acid;

or a pharmaceutically acceptable salt thereof.

The compounds of Formula (1) are generally and specifically described in published PCI patent application WO2010/020675, which is hereby incorporated by reference.

### Second General Embodiment of the Invention

A combination comprising a first agent that is a cyclin dependent kinase 4/6(CDK4/6) inhibitor and a second agent that is an mTOR inhibitor, wherein the first agent is a compound of Formula II:

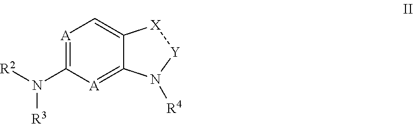

or a pharmaceutically acceptable salt or solvate thereof, wherein:

the dashed line indicates a single or double bond;

A is N or CR⁵, Wherein R⁵ is hydrogen or C₁-C₃-alkyl;

R² and R³ are each, independently, selected from the group consisting of hydrogen, hydroxyl, C₁-C₃-alkyl, C₃-C₈-cycloalkyl, heterocyclyl, aryl, heteroaryl, substituted C₁-C₃-alkyl, substituted C₃-C₈-cycloalkyl, substituted heterocyclyl, substituted aryl and substituted heteroaryl;

R⁴ is selected from the group consisting of hydrogen, C₁-C₈-alkyl, substituted C₁-C₈-alkyl, substituted C₃-C₈-cycloalkyl, aryl, substituted aryl, heteroaryl and substituted heteroaryl;

when the bond between X and Y is a single bond, X is CR⁶R⁷, NR⁸ or C═O, and Y is CR⁹R¹⁰or C═O;

when the bond between X and Y is a double bond, X is N or CR¹¹, and Y is CR¹²;

wherein R⁶ and R⁷ are each, independently selected from the group consisting of aryl, substituted aryl, heteroaryl, substituted heteroaryl, hydrogen, C₁-C₃-alkyl, C₃-C₈-cycloalkyl, heterocyclyl, substituted alkyl, substituted cycloalkyl, and substituted heterocyclyl;

R⁸ is hydrogen, C₁-C₃-alkyl, and C₃-C₈-cycloalkyl;

R⁹ and R¹⁰ are each, independently, hydrogen, C₁-C₃-alkyl, or C₃-C₈-cycloalkyl;

R¹¹ and R¹² are each, independently, selected from the group consisting of halo, hydrogen, C₁-C₃-alkyl, C₁-C₃-alkoxy, CN, C═NOH, C═NOCH₃, C(O)H, C(O)C₁-C₃-alkyl, C₃-C₈-cycloalkyl, heterocyclyl, aryl, heteroaryl, substituted C₁-C₃-alkyl, substituted C₃-C₈-cycloalkyl, substituted heterocyclyl, substituted aryl, substituted heteroaryl, —BNR¹³R¹⁴, —BOR¹³, —BC(O)R¹³, —BC(O)OR¹³, —BC(O)NR¹³R¹⁴; wherein B is a bond, C₁-C₃-alkyl or branched C₁-C₃alkyl; wherein R¹³ and R¹⁴ are each, independently, selected from the group consisting of hydrogen, C₁-C₃-alkyl, C₃-C₈-cycloalkyl, heterocyclyl, aryl, heteroaryl, substituted alkyl, substituted cycloalkyl, substituted heterocyclyl, substituted aryl, and substituted heteroaryl.

In one embodiment of the second general embodiment, the compound of Formula II is selected from the group consisting of

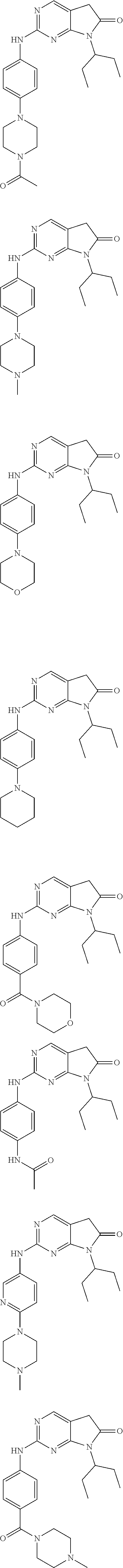

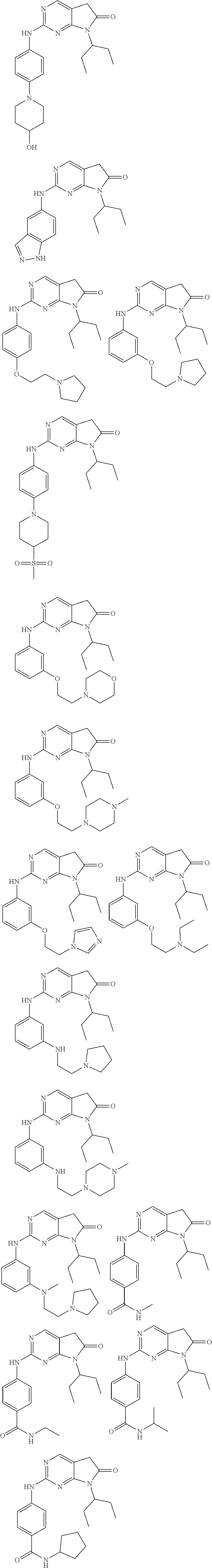

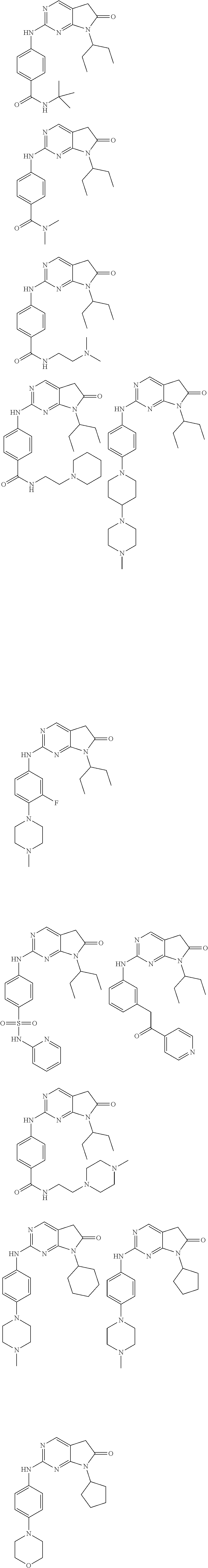

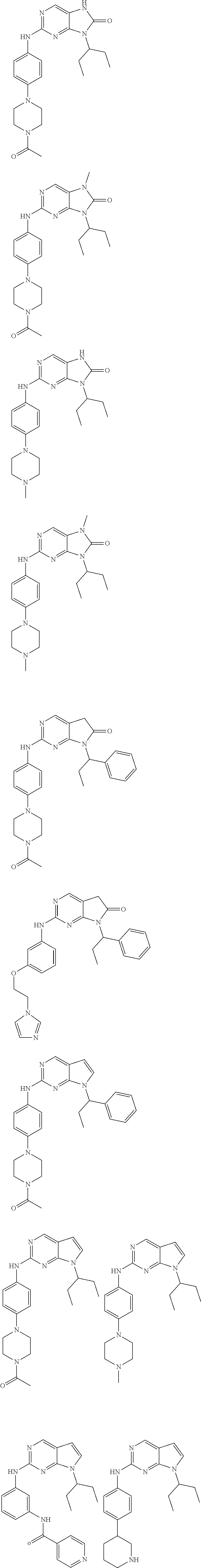

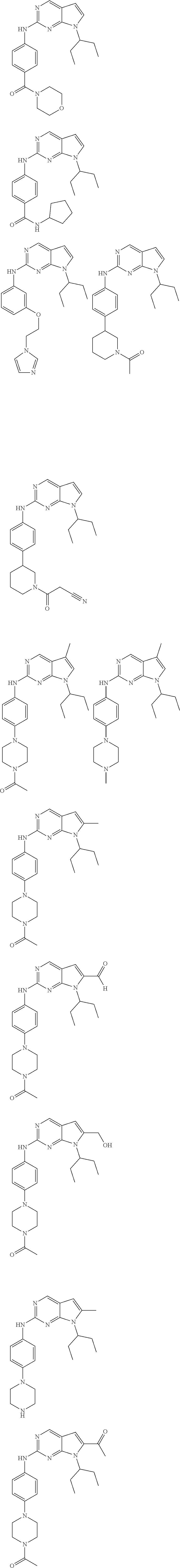

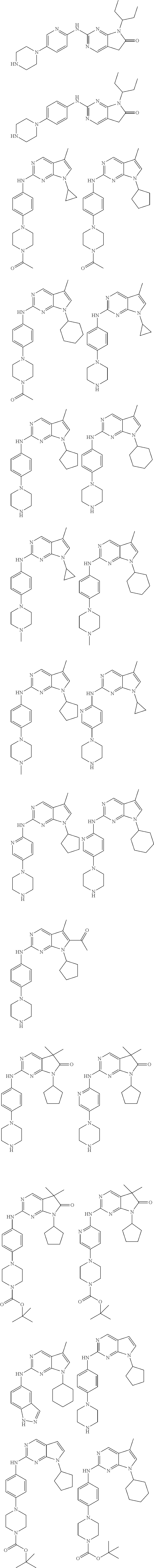

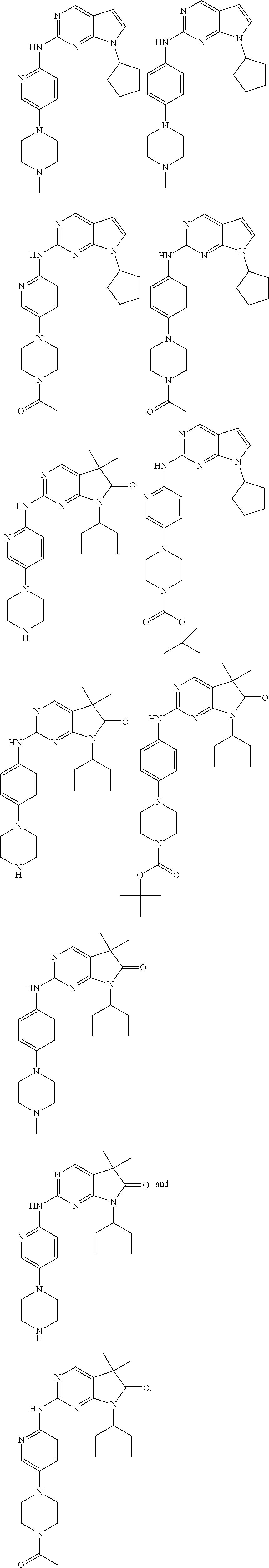

The compounds of Formula II are generally and specifically described in published PCT patent application WO20071140222, which is hereby incorporated by reference.

### Third General Embodiment of the Invention

A combination comprising a first agent that is a cyclin dependent kinase 4/6(CDK4/6) inhibitor and a second agent that is an mTOR inhibitor, wherein the first agent is a compound of Formula III:

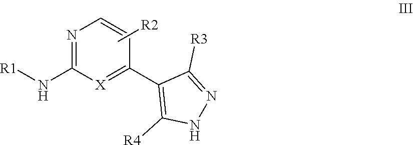

or a pharmaceutically acceptable salt, Wherein
R¹ is C₁₋₆-alkyl, C₃₋₁₄-cycloalkyl, a 3-14 membered cycloheteroalkyl group, C₆₋₁₄aryl, C₁₋₆-alkoxy, C₁₋₆alkyC₆₋₁₄aryl, C₁₋₆alkylC₃₋₁₄cycloalkyl, C₁₋₆alkyl-3-14 membered cycloheteroalkyl group, C₁₋₆alkyl-5-14 membered heteroaryl group, C₁₋₆alkylOR⁷, C₁₋₆-alkylNR⁵R⁶, C₁₋₆alkoxyC₆₋₁₄aryl, C₁₋₆alkylCN, or C₁₋₆alkylC(O)OR⁷, which may be unsubstituted or substituted with one or more of C₁₋₆-alkyl, C₆₋₁₄-aryl, hydroxyl, C₁₋₆-alkylhalo, C₁₋₆alkoxyhalo, halo, C₁₋₆-alkoxy, C₁₋₆alkyC₆₋₁₄aryl, C(O)OR⁸, CN, oxo, or NR⁹R¹⁰;
R² is H, C₁₋₆alkyl, C₂₋₆-alkenyl, C₂₋₆-alkynyl, hydroxyl, or halo;
R³ and R⁴ are independently C₃₋₁₄-cycloalkyl, or halo, which may be unsubstituted or substituted;
R⁵, R⁶, R⁷, R⁸, R⁹ , and R¹⁰ independently are hydrogen, C₁₋₆-alkyl, C₂₋₆-alkenyl, C₂₋₆-alkynyl, C₃₋₁₄-cycloalkyl, a 5 -14 membered heteroaryl group, C₆₋₁₄-aryl, C(O)OR¹¹, or C(O)R¹¹, which may be unsubstituted or substituted;

X is N or CR¹² where R¹¹ and R¹² are independently H, halogen, or C₁₋₆-alkyl.

In one embodiment of the third general embodiment, the compound of Formula III wherein R¹ is C₁₋₆-alkyl, C₃₋₁₄-cycloalkyl, C₆₋₁₄aryl, a 3-14 membered cycloheteroalkyl group, C₁₋₆alkyC₆₋₁₄aryl, C₁₋₆alkylC₃₋₁₄cycloalkyl, C₁₋₆alkyl-3-14 membered cycloheteroalkyl group, or C₁₋₆alkyl-5-14 membered heteroaryl group, which may be unsubstituted or substituted with one or more of C₁₋₆-alkyl, C₆₋₁₄-aryl, hydroxyl, C₁₋₆-alkylhalo, halo, C₁₋₆-alkoxy, C₁₋₆alkyC₆₋₁₄aryl.

Examples of compounds of Formula III include

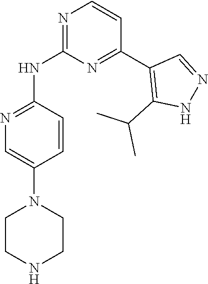 [SMILES: CC(C)c1[nH]ncc1-c1ccnc(Nc2ccc(N3CCNCC3)cn2)n1]

([4-(5-Isopropyl-1H-pyrazol-4-yl)-pyrimidin-2-yl]-(5-piperazin-1-yl-pyridin-2-yl)-amine) and

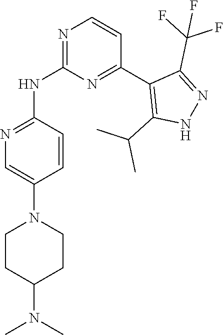 [SMILES: CC(C)c1[nH]nc(C(F)(F)F)c1-c1ccnc(Nc2ccc(N3CCC(N(C)C)CC3)cn2)n1]

(N*6′*-[4-(5-isopropyl-3-trifluoromethyl-1H-pyrazol-4-yl)-pyrimidin-2-yl]-N*4*,N*4*-dimethyl-3,45,6-tetrahydro-2H-[1,3′]bipyridinyl-4,6′-diamine).

The compounds of Formula III are generally and specifically described in published PCT patent application WO2009/071701, which is hereby incorporated by reference.

### Fourth General Embodiment of the Invention

A combination comprising a first agent that is a cyclin dependent kinase 4/6(CDK4/6) inhibitor and a second agent that is an mTOR inhibitor, wherein the first agent is a compound of Formula IV:

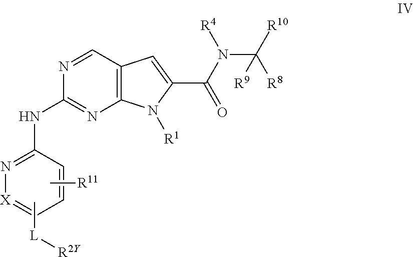

wherein:
R¹ is C₃₋₇ alkyl; C₄₋₇ cycloalkyl optionally substituted with one substituent selected from the group consisting of C₁₋₆ alkyl and OH; phenyl optionally substituted with one substitutent selected from the group consisting of C₁₋₆ alkyl, C(CH₃)₂CN, and OH; piperidinyl optionally substituted with one cyclopropyl or C₁₋₆ alkyl; tetrahydropyranyl optionally substituted with one cyclopropyl or C₁₋₆ alkyl; or bicyclo[2.2.1]heptanyl;
A is CH or N;
R¹¹ is hydrogen or C₁₋₄ alkyl;
L is a bond, C(O), or S(O)₇;

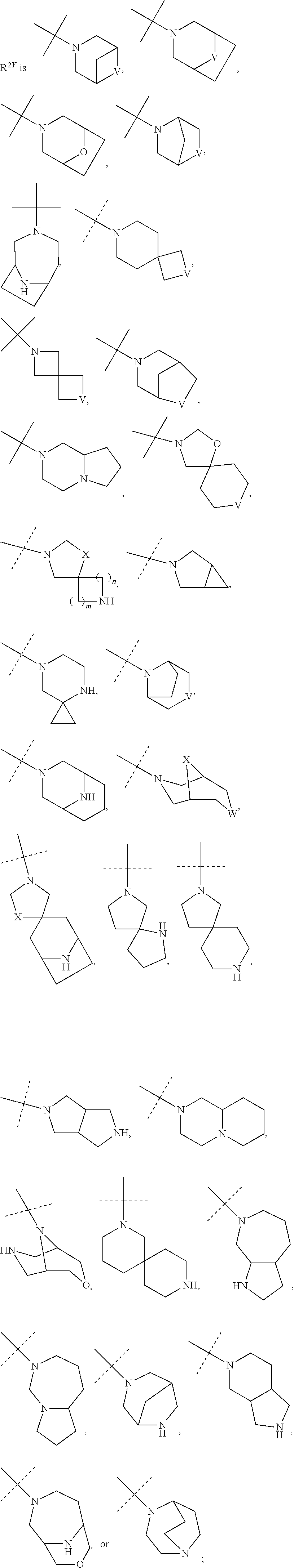

V is NH or CH₂;
X is O or CH₂;
W is O or NH;
m and n are each independently 1, 2, or 3 provided that m and n are not both 3;
each R^{2Y} is optionally substituted with one to four substituents each independently selected from the group consisting of: C₁₋₃ alkyl optionally substituted with one or two substituents each independently selected from the group consisting of hydroxy, NH₂, and —S—C₁₋₃ alkyl; CD₃; halo; oxo; C₁₋₃ haloalkyl; hydroxy; NH₂; dimethylamino; benzyl; —C(O)-C₁₋₃alkyl optionally substituted with one or two substituents each independently selected from the group consisting of NH_{2′} —SCH₃ and NHC(O)CH₃; —S(O)₂—C₁₋₄alkyl; pyrrolidinyl-C(O)—; and —C(O)₂—C_{1□3}alkyl;
R⁴ is hydrogen, deuterium, or C(R⁵)(R⁶)(R⁷); and
R⁵, R⁶, R⁷, R⁸, R⁹ and R¹⁰ are each independently H or deuterium; or a pharmaceutically acceptable salt thereof.

In one embodiment of the fourth general embodiment, cyclin dependent kinase 4/6(CDK4/6) inhibitor is a compound described by Formula IV-B:

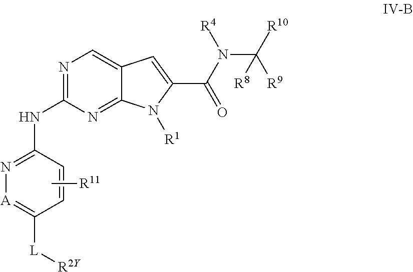

wherein
L is a bond or C(O);

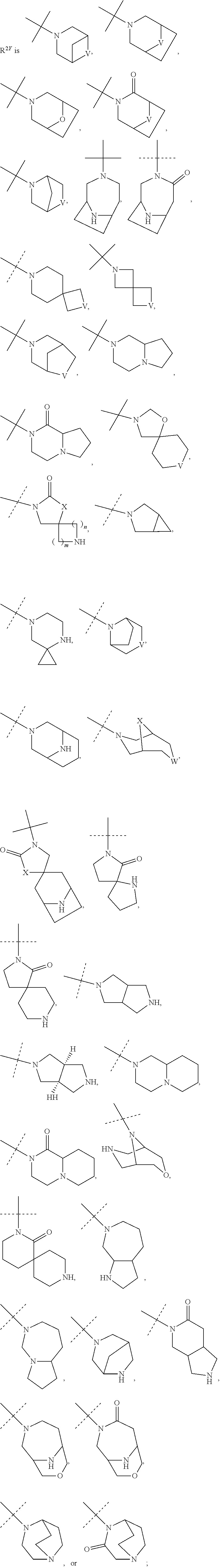

V is NH or CH₂;
X is O or CH₂;
W is O or NH;
m and n are each independently 1, 2, or 3 provided that m and n are not both 3; and each R⁵ is optionally substituted with one to four substituents each independently selected from the group consisting of: C₁₋₃ alkyl optionally substituted with one or two substituents each independently selected from the group consisting of hydroxy, NH₂, and —S—C₁₋₃ alkyl; CD₃; C₁₋₃ haloalkyl; hydroxy; NH₂; dimethylamino; benzyl; —C(O)—C₁₋₃alky) optionally substituted with one or two substituents each independently selected from the group consisting of NH_{2′} —SCH₃ and NHC(O)CH₃; —S(O)₂—C₁₋₄alkyl; pyrrolidinyl-C(O)—; and —C(O)₂—C_{1□3}alkyl; or a pharmaceutically acceptable salt thereof.

The compounds of Formula IV are generally and specifically described in pending PCT application PCT/EP2011/052353, which is hereby incorporated by reference.

### Fifth General Embodiment of the Invention

A combination comprising a first agent that is a cyclin dependent kinase 4/6(CDK4/6) inhibitor and a second agent that is an mTOR inhibitor, wherein the first agent is a compound of Formula V:

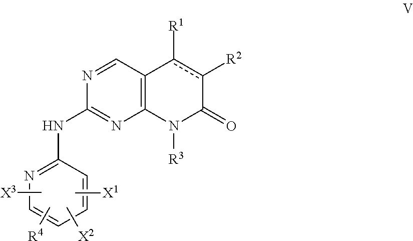

wherein:
the dashed line represents an optional bond,
X¹, X², and X³ are in each instance independently selected from hydrogen, halogen, C₁-C₆ alkyl, C₁-C₆ haloalkyl, C₁-C₈ alkoxy, C₁-C₈ alkoxyalkyl, CN, NO₂, OR⁵, NR⁵R⁶, CO₂R⁵, COR⁵, S(O)NR⁵, CONR⁵R⁶, NR⁵COR⁶, NR⁵SO₂R⁶, SO₂NR⁵R⁶, and
P(O)(OR⁵)(OR⁶); with the proviso that at least one of X¹, X², and X³ must be hydrogen;
n=0-2;
R¹ is, in each instance, independently, hydrogen, halogen, C₁-C₆ alkyl, C₁-C₆ haloalkyl, C₁-C₆ hydoxyalkyl, or C₃-C₇ cycloalkyl;

R² and R⁴ are independently selected from hydrogen, halogen, C₁-C₈ alkyl, C₃-C₇ cycloalkyl, C₁-C₈ alkoxy, C₁-C₈ alkoxyalkyl, C₁-C₈ haloalkyl, C₁-C₈ hydroxyalkyl C₂-C₈ alkenyl, C₂-C₈ alkynyl, nitrile, nitro, OR⁵, SR⁵, NR⁵R⁶, N(O)R⁵R⁶, P(O)(OR⁵)(OR⁶), (CR⁵R⁶)ₘNR⁷R⁸, COR⁵, (CR⁴R⁵)ₘC(O)R⁷, COR₂, CONR⁵R⁶, C(O)NR⁵SO₂R⁶, NR⁵SO₂R⁶, C(O)NR⁵OR⁶, S(O)ₘR⁵, SO₂NR⁵R⁶, P(O)(OR⁵)(OR⁶), (CR⁵R⁶)ₘP(O)(OR⁷)(OR⁸)—, (CR⁵R⁶)ₘ-aryl (CR⁵R⁶)ₘ-heteroaryl, T(CH₂)ₘQR⁵, —C(O)T(CH₂)ₘQR⁵, NR⁵C(O)T(CH₂)ₘQR⁵, and —CR⁵═CR⁶C(O)R⁷; or
R¹ and R² may form a carbocyclic group containing 3-7 ring members, preferably 5-6 ring members, up to four of which can optionally be replaced with a heteroatom independently selected from oxygen, sulfur, and nitrogen, and wherein the carbocyclic group is unsubstituted or substituted with one, two, or three groups independently selected from halogen, hydroxy, hydroxyalkyl nitrile, lower C₁-C₈ alkyl, lower C₁-C₈ alkoxy, alkoxycarbonyl, alkylcarbonyl, alkylcarbonylamino, aminoalkyl, trifluoromethyl, N-hydroxyacetamide, trifluoromethylalkyl, amino, and mono or dialkylamino, (CH₂)ₘC(O)NR⁵R⁶, and O(CH₂)ₘC(O)OR⁵, provided, however, that there is at least one carbon atom in the carbocyclic ring and that if there are two or more ring oxygen atoms, the ring oxygen atoms are not adjacent to one another;
T is O, S, NR⁷, N(O)R⁷, NR⁷R⁸W, or CR⁷R⁸;
Q is O, S, NR⁷, N(O)R⁷, NR⁷R⁸W, CO₂, O(CH₂)ₘ-heteroaryl, O(CH₂)ₘS(O)ₙR⁸, (CH₂)-heteroaryl, or a carbocyclic group containing from 3-7 ring members, up to four of which ring members are optionally heteroatoms independently selected from oxygen, sulfur, and nitrogen, provided, however, that there is at least one carbon atom in the carbocyclic ring and that if there are two or more ring oxygen atoms the ring oxygen atoms are not adjacent to one another, wherein the carbocyclic group is unsubstituted or substituted with one, two, or three groups independently selected from halogen, hydroxy, hydroxyalkyl, lower alkyl, lower alkoxy, alkoxycarbonyl, alkylcarbonyl, alkylcarbonylamino, aminoalkyl, trifluoromethyl, N-hydroxyacetamide, trifluoromethylalkyl, amino, and mono or dialkylamino;
W is an anion selected from the group consisting of chloride, bromide, trifluoroacetate, and triethylammonium;
m=0-6;
R⁴ and one of X¹, X² and X³ may form an aromatic ring containing up to three heteroatoms independently selected from oxygen, sulfur, and nitrogen, and optionally substituted by up to 4 groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, mono- or dialkylamino, N-hydroxyacetamido, aryl, heteroaryl, carboxyalkyl, nitrite, NR⁷SO₂R⁸, C(O)NR⁷R⁸, NR⁷C(O)R⁸, C(O)ₙR⁷, C(O)NR⁷SO₂R⁸, (CH₂)ₘS(O)₋ₙR⁷, (CH₂)ₘ heteroaryl, O(CH₂)ₘ-heteroaryl, (CH₂)ₘC(O)NR⁷R⁸, O(CH₂)ₘC(O)OR⁷, (CH₂)ₘSO₂NR⁷R⁸, and C(O)R⁷;
R³ is hydrogen, aryl, C₁-C₈ alkyl, C₁-C₈ alkoxy, C₃-C₇ cycloalkyl, or C₃-C₇-heterocyclyl;
R⁵ and R⁶ independently are hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl; or
R⁵ and R⁶, when attached to the same nitrogen atom, taken together with the nitrogen to which they are attached, form a heterocyclic ring containing from 3-8 ring members, up to four of which members can optionally be replaced with heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl, NR⁷SO₂R⁸, C(O)NR⁷R⁸, NR⁷C(O)R⁸, C(O)OR⁷, C(O)NR⁷SO₂R⁸, (CH₂)ₘS(O)ₙR⁷, (CH₂)ₘ-heteroaryl, O(CH₂)ₘ-heteroaryl, (CH₂)ₘC(O)NR⁷R⁸, O(CH₂)ₘC(O)OR⁷, and (CH₂)SO₂NR⁷R⁸;
R⁷ and R⁸ are, independently, hydrogen, C₁-C₈ alkyl, C₂-C₈ alkenyl, C₂-C₈ alkynyl, arylalkyl, cycloalkyl, heterocycloalkyl, aryl, heteroaryl, or heterarylalkyl; or
R⁷ and R⁸, when attached to the same nitrogen atom, taken together with the nitrogen to which they are attached, may form a heterocyclic ring containing from 3-8 ring members, up to four of which members are optionally heteroatoms independently selected from oxygen, sulfur, S(O), S(O)₂, and nitrogen, provided, however, that there is at least one carbon atom in the heterocyclic ring and that if there are two or more ring oxygen atoms, the ring oxygen atoms are not adjacent to one another, wherein the heterocyclic group is unsubstituted or substituted with one, two or three groups independently selected from halogen, hydroxy, hydroxyalkyl, lower alkyl, lower alkoxy, alkoxycarbonyl, alkylcarbonyl, alkylcarbonylamino, aminoalkyl, aminoalkylcarbonyl, trifluoromethyl, trifluoromethylalkyl, trifluoromethylalkylaminoalkyl, amino, nitrile, mono- or dialkylamino, N-hydroxyacetamido, aryl, heteroaryl, carboxyalkyl; and the pharmaceutically acceptable salts, esters, amides, and prodrugs thereof.

The compounds of Formula V are generally and specifically described in published PCT patent application WO 2003/062236, which is hereby incorporated by reference.

In addition of the first through fifth general embodiments, the present invention also relates to a combination comprising a first agent that is a cyclin dependent kinase 4/6(CDK(4/6) inhibitor and a second agent that is an mTOR inhibitor, wherein the first agent is a compound is generally and specifically described in published PCT patent application WO2010/125402, which is hereby incorporated by reference or a compound generally and specifically described in published PCT patent application WO2008/007123, which is hereby incorporated by reference.

Specific exemplary cyclin dependent kinase 4/6(CDK4/6) inhibitors include, but not limited to:

Compound A1: 7-Cyclopentyl-2-(5-piperazin-1-yl-pyridin-2-ylamino)-7H-pyrrolo[2,3-d]pyrimidine-6-carboxylic acid dimethylamide, which has the following chemical structure

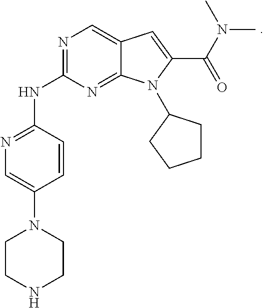 [SMILES: CN(C)C(=O)c1cc2cnc(Nc3ccc(N4CCNCC4)cn3)nc2n1C1CCCC1]

Compound A2: 7-Cyclopentyl-2-[5-(3,8-diaza-bicyclo[3.2.1.]octane-3-carbonyl)-pyridin-2-ylamino]-7H-pyrrolo[2,3-d]pyrimidine-6-carboxylic acid dimethylamide, which has the following chemical structure:

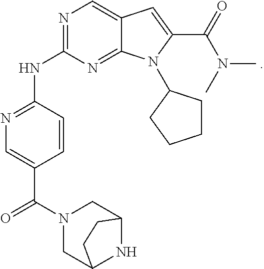 [SMILES: CN(C)C(=O)c1cc2cnc(Nc3ccc(C(=O)N4CC5CCC(C4)N5)cn3)nc2n1C1CCCC1]

Compound A3: 7-Cyclopentyl-2-[5-((1R,6S)-9-methyl-4-oxo-3,9-diaza-bicyclo[4.2.1]-non-3-yl)-pyridin-2-ylamino]-7H-pyrrolo[2,3-d]pyrimidine-6-carboxylic acid dimethylamide, which has the following chemical structure:

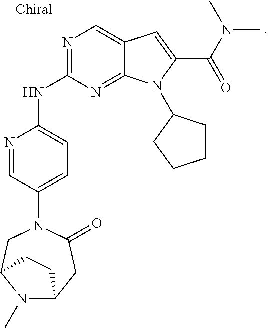 [SMILES: CN(C)C(=O)c1cc2cnc(Nc3ccc(N4C[C@H]5CC[C@@H](CC4=O)N5C)cn3)nc2n1C1CCCC1]

Compound A4: 6-Acetyl-8-cyclopentyl-5-methyl-2-(5-piperazin-1-yl-pyridin-2-ylamino)-8H-pyrido[2,3-d]pyrimidin-7-one, which has the following chemical structure:

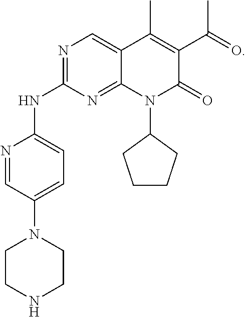 [SMILES: CC(=O)c1c(C)c2cnc(Nc3ccc(N4CCNCC4)cn3)nc2n(C2CCCC2)c1=O]

Compound A5: N*6′*[4-(5-Isopropyl-3-trifluoromethyl-1H-pyrazol-4-yl]-N*4*,N*4*-dimethyl-3,4,5,6-tetrahydro-2H-[1,3′]bipyridinyl-4,6′-diamine, which has the following chemical structure:

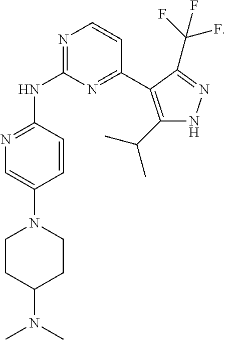 [SMILES: CC(C)c1[nH]nc(C(F)(F)F)c1-c1ccnc(Nc2ccc(N3CCC(N(C)C)CC3)cn2)n1]

Compound A6: [4-(5-Isopropyl-1H-pyrazol-4-yl)-pyrimidin-2-yl]-(5-piperazin-1-yl-pyridin-2-yl)-amine, which has the following chemical structure:

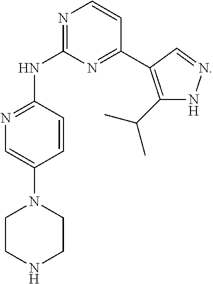 [SMILES: CC(C)c1[nH]ncc1-c1ccnc(Nc2ccc(N3CCNCC3)cn2)n1]

Exemplary mTOR inhibitors which may be used to practice the invention, include Sirolimus (rapamycin, AY-22989, Wyeth), Everolimus (RAD001, Novartis), Temsirolimus (CCI-779, Wyeth) and Deferolimus (AP-23573/MK-8669, Ariad/Merck & Co), AP23841 (Ariad) AZD-8055 (AstraZeneca), Ku-0063794 (AstraZeneca, Kudos), OSI-027 (OSI Pharmaceuticals), WYE-125132 (Wyeth), Zotarolimus (ABT-578), SAR543, Ascomycin, INK-128 (Intellikine) XL765 (Exelisis), NV-128 (Novogen), WYE-125132 (Wyeth), EM101/LY303511 (Emiliem), {5-[2,4-Bis-((S)-3-methyl-morpholin-4-yl)-pyrido[2,3-d]pyrimidin-7-yl]-2-methoxy-phenyl}-methanol), the compound OSI-027 (OSI)

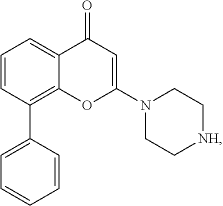 [SMILES: O=c1cc(N2CCNCC2)oc2c(-c3ccccc3)cccc12]

### HTS-1 (University of Leicester)

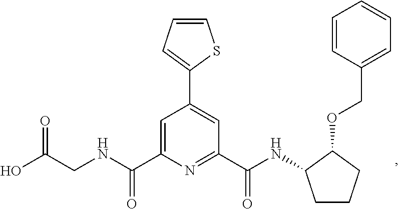 [SMILES: O=C(O)CNC(=O)c1cc(-c2cccs2)cc(C(=O)N[C@H]2CCC[C@H]2OCc2ccccc2)n1]

### and PP242 (Intellikine)

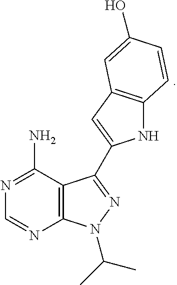 [SMILES: CC(C)n1nc(-c2cc3cc(O)ccc3[nH]2)c2c(N)ncnc21]

Each of the mTOR inhibitors described above can be used in combination with any of the general and/or specific embodiments of the cyclin dependent kinase 4/6(CDK4/6) inhibitor described above.

Everolimus, which is Compound B1, has the chemical name ((1R,9S,12S,15R,16E,18R,19R,21R,23S,24E,26E,28E,30S,32S,35R)-1,18-dihydroxy-12-{(1R)-2-[(1S,3R,4R)-4-(2-hydroxyethoxy)-3-methoxycyclohexyl]-1-methylethyl}-19,30-dimethoxy-15,17,21,23,29,35-hexamethyl-11,36-dioxa-4-aza-tricyclo[30.3.1.04,9] hexatriaconta-16,24, 26,28-tetraene-2,3,10,14,20-pentaone.) Everolimus and analogues are described in U.S. Pat. No. 5,665,772, at column 1, line 39 to column 3, line 11. Everolimus is described by the following structure:

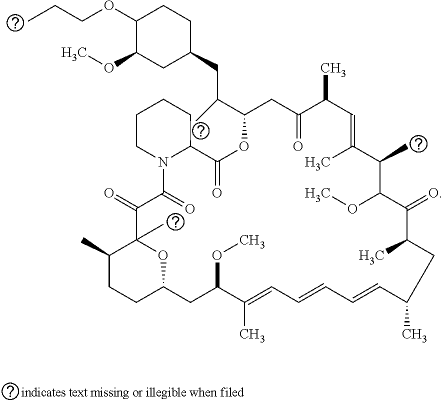 [SMILES: CCCOC1CC[C@@H](CC(C)[C@@H]2CC(=O)[C@@H](C)/C=C(\C)[C@@H](C)C(OC)C(=O)[C@H](C)C[C@H](C)/C=C/C=C/C=C(\C)[C@H](OC)C[C@@H]3CC[C@@H](C)C(C)(O3)C(=O)C(=O)N3CCCCC3C(=O)O2)C[C@H]1OC]

Rapamycin, which is Compound B2, has the chemical name (3S,6R,7E,9R,10R,12R,14S,15E,17E,19E,21S,23S, 26R,27R,34aS)-9,10,12,13,14,21,22,23,24,25,26,27,32,33,34,34a-hexadecahydro-9,27-dihydroxy-3-[1R)-2-[(R1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4]-oxaazacyclohentriacontine-1,5,11,28,29(4H,6H,31H)-pentone. It is described by the following structure:

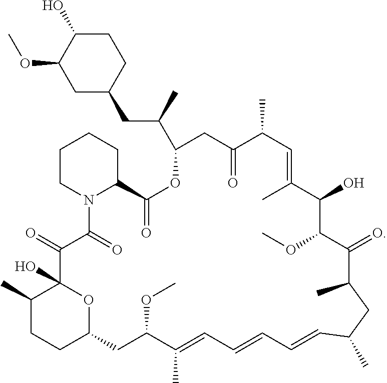

Other mTOR inhibitors useful with the present invention include those disclosed in US Patent Application Publication Nos. 2008/0194546 and 2008/0081809, the compounds described in the examples of WO 06/090167; WO 06/090169; WO 07/080382, WO 07/060404, WO07/061737 and WO07/087395 and WO08/02316, and the compounds described in J. Med. Chem.. 2009, 52, 5013-5016.

In another embodiment, the present invention includes a combination where said second agent is selected from the group consisting of rapamycin (AY-22989), everolimus, CCI-779, AP-23573, MK-8669, AZO-8055, Ku-0063794,OSI-027, WYE-125132. In a preferred embodiment the second agent is everolimus.

In another embodiment of the present invention, the inhibitor of mTOR is selected from Rapamycin derivatives such as:

a. substituted rapamycin e.g. a 40-O-substituted rapamycin e.g. the compounds described in U.S. Pat. No. 5,258,389, WO 94/09010, WO 92/05179, U.S. Pat. No. 5,118,677, U.S. Pat. No. 5,118,678, U.S. Pat. No. 5,100,883, U.S. Pat. No. 5,151,413, U.S. Pat. No. 5,120,842, WO 93/11130, WO 94/02136, WO 94/02485 and WO 95/14023;

b. a 16-O-substituted rapamycin e.g. the examples disclosed in WO 94/02136, WO 95/16691 and WO96/41807;

c. a 32-hydrogenated rapamycin e.g. the examples disclosed in WO 96/41807 and U.S. Pat. No. 5,256,790;

d. derivatives disclosed in WO 94/09010, WO 95/16691 or WO 96/41807, more suitably 32-deoxorapamycin, 16-pent-2-ynyloxy-32-deoxorapamycin 16-pent-2-ynyloxy-32(S)-dihydro-rapamycin, 16-pent-2-ynyloxy-32(S)-dihydro-40-O-(2-hydroxyethyl)-rapamycin and, more preferably, 40-O-(2-hydroxyethyl)-rapamycin, disclosed as Example 8 in WO 94/09010, preferably 40-O-(2-hydroxyethyl)-rapamycin, 40-[3-hydroxy-2-(hydroxymethyl)-2-methylpropanoate]-rapamycin (also called CCI-779), 40-epi-(tetrazolyl)-rapamycin (also called ABT578), 32-deoxorapamycin, 16-pent-2-ynyloxy-32(S)-dihydro rapamycin, or TAFA-93; and

e. derivatives disclosed in WO 98/02441 and WO 01/14387, e.g. AP23573, AP23464, or AP23841.

In yet another embodiment, the present invention includes a combination where said second agent is selected from the group consisting of AY-22989, everolimus, CCI-779, AP-23573, MK-8669, AZD-8055, Ku-0063794, OSI-027, WYE-125132. In a preferred embodiment the second agent is everolimus.

In another embodiment, the present invention includes a method of treating a hyperproliferative disease, preferably cancer, dependent on CDK4/6 or mTOR, the method comprising administering to a patient in need thereof a combination of the present invention. CDK4/6 dependent cancers are also generally marked by a hyperphosphorlyated (retinoblastoma) Rb protein. A cancer is dependent on a pathway if inhibiting or blocking that pathway will slow or disrupt growth of that cancer. Examples of CDK4 or CDK6 pathway dependent cancers include breast cancer, non small cell lung cancer, melanoma, colon cancer, esophageal cancer, liposarcoma, mantle cell lyomphoma, multiple myeloma, T-cell leukemia, renal cell carcinoma, gastric cancer and pancreatic cancer. Examples of mTOR pathway dependent cancers include breast cancer, pancreatic cancer, renal cell carcinoma, mantle cell lymphoma, glioblastorna, hepatocellular carcinoma, gastric cancer, lung cancer and colon cancer. Correlation of cancers with the CDK4/6 pathway or the mTOR pathway has been established in the art. For example, see Shapiro, Journal of Clinical Oncology, Vol. 24, No. 11 (2006) pp. 1770-1783 or Fasolo, Expert Opin. Investig. Drugs Vol. 17, No. 11 (pp. 1717-1734.

Therefore in an embodiment of the invention is a combination of a CDK4/6 inhibitor and an mTOR inhibition for use in treating cancer, by manufacture in a medicament, which can be sold as either a combine or separate dosage form, or a method of treating cancer by administering the combination to a patient in need thereof. The cancer can be a solid tumor cancer or a lymphoma. Preferred cancers include pancreatic cancer, breast cancer, mantle cell lyomphoma, non small cell lung cancer, melanoma, colon cancer, esophageal cancer, liposarcoma, multiple myeloma, T-cell leukemia, renal cell carcinoma, gastric cancer, renal cell carcinoma, glioblastoma, hepatocellular carcinoma, gastric cancer, lung cancer or colon cancer.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The phrase “therapeutically effective amount” is used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition/symptom in the host.

“Agent” refers to all materials that may be used to prepare pharmaceutical and diagnostic compositions, or that may be compounds, nucleic acids, polypeptides, fragments, isoforms, variants, or other materials that may be used independently for such purposes, all in accordance with the present invention.

“Analog” as used herein, refers to a small organic compound, a nucleotide, a protein, or a polypeptide that possesses similar or identical activity or function(s) as the compound, nucleotide, protein or polypeptide or compound having the desired activity and therapeutic effect of the present invention. (e.g., inhibition of tumor growth), but need not necessarily comprise a sequence or structure that is similar or identical to the sequence or structure of the preferred embodiment.

“Derivative” refers to either a compound, a protein or polypeptide that comprises an amino acid sequence of a parent protein or polypeptide that has been altered by the introduction of amino acid residue substitutions, deletions or additions, or a nucleic acid or nucleotide that has been modified by either introduction of nucleotide substitutions or deletions, additions or mutations. The derivative nucleic acid, nucleotide, protein or polypeptide possesses a similar or identical function as the parent polypeptide.

As used herein, “halo” or “halogen” refers to fluoro, chloro, bromo, and iodo.

As used herein, “alkyl” refers to a straight-chain or branched saturated hydrocarbon group. In some embodiments, an alkyl group can have from 1 to 10 carbon atoms (e.g., from 1 to 8 carbon atoms). Examples of alkyl groups include methyl (Me), ethyl (Et), propyl (e.g., n-propyl and isopropyl), butyl (e.g., n-butyl, isobutyl, s-butyl, t-butyl), pentyl groups n-pentyl, isopentyl, neopentyl), hexyl (e.g., n-hexyl and its isomers), and the like. A lower alkyl group typically has up to 4 carbon atoms. Examples of lower alkyl groups include methyl, ethyl, propyl (e.g., n-propyl and isopropyl), and butyl groups (e.g., isobutyl, s-butyl, t-butyl). In an embodiment an alkyl group, or two or more alkyl groups may form a bridged alkyl group. This is where an alkyl group links across another group (particularly shown in cyclic groups), forming a ring bridged by an alkyl chain, i.e., forming a bridged fused ring. This is shown, but not limited to where two or more le groups for a bridged alkyl group across the Y ring group forming a ring bridged by an alkyl chain.

As used herein, “alkenyl” refers to a straight-chain or branched alkyl group having one or more carbon-carbon double bonds. In some embodiments, an alkenyl group can have from 2 to 10 carbon atoms (e.g., from 2 to 8 carbon atoms). Examples of alkenyl groups include ethenyl, propenyl, butenyl, pentenyl, hexenyl, butadienyl, pentadienyl, hexadienyl groups, and the like. The one or more carbon-carbon double bonds can be internal (such as in 2-butene) or terminal (such as in 1-butene).

As used herein, “alkynyl” refers to a straight-chain or branched alkyl group having one or more carbon-carbon triple bonds. In some embodiments, an alkynyl group can have from 2 to 10 carbon atoms (e.g., from 2 to 8 carbon atoms). Examples of alkynyl groups include ethynyl, propynyl butynyl, pentynyl, and the like. The one or more carbon-carbon triple bonds can be internal (such as in 2-butyne) or terminal (such as in 1-butyne).

As used herein, “alkoxy” refers to an —O-alkyl group. Examples of alkoxy groups include methoxy, ethoxy, propoxy (e.g., n-propoxy and isopropoxy), t-butoxy groups, and the like.

As used herein, “alkylthio” refers to an —S-alkyl group. Examples of alkylthio groups include methylthio, ethylthio, propylthio (e.g., n-propylthio and isopropylthio), t-butylthio groups, and the like.

The term “carbalkoxy” refers to an alkoxycarbonyl group, where the attachment to the main chain is through the carbonyl group (C(O)). Examples include but are not limited to methoxy carbonyl, ethoxy carbonyl, and the like.

As used herein, “oxo” refers to a double-bonded oxygen (i.e., ═O). It is also to be understood that the terminology C(O) refers to a —C═O group, whether it be ketone, aldehyde or acid or acid derivative. Similarly, S(O) refers to a —S═O group.

As used herein, “haloalkyl” refers to an alkyl group having one or more halogen substituents. In some embodiments, a haloalkyl group can have 1 to 10 carbon atoms (e.g., from 1 to 8 carbon atoms). Examples of haloalkyl groups include CF₃, C₂F₅, CHF₂, CH₂F, CCl₃, CHCl₂, CH₂Cl, C₂Cl₅, and the like. Perhaloalkyl groups, i.e., alkyl groups wherein all of the hydrogen atoms are replaced with halogen atoms (e.g., CF₃ and C₂F₅), are included within the definition of “haloalkyl.” For example, a C₁₋₁₀ haloalkyl group can have the Formula —CᵢH₂ᵢ₊₁₋ⱼXⱼ, wherein X is F, CI, Br, or I, i is an integer in the range of 1 to 10, and j is an integer in the range of 0 to 21, provided that j is less than or equal to 2i+1.

As used herein, “cycloalkyl” refers to a non-aromatic carbocyclic group including cyclized alkyl, alkenyl, and alkynyl groups. A cycloalkyl group can be monocyclic (e.g., cyclohexyl) or polycyclic (e.g., containing fused, bridged, and/or Spiro ring systems), wherein the carbon atoms are located inside or outside of the ring system. A cycloalkyl group, as a whole, can have from 3 to 14 ring atoms (e.g., from 3 to 8 carbon atoms for a monocyclic cycloalkyl group and from 7 to 14 carbon atoms for a polycyclic cycloalkyl group). Any suitable ring position of the cycloalkyl group can be covalently linked to the defined Chemical structure. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcaryl, adamantyl, and spiro[4.5]decanyl groups, as well as their homologs, isomers, and the like.

As used herein, “heteroatom” refers to an atom of any element other than carbon or hydrogen and includes, for example, nitrogen, oxygen, sulfur, phosphorus, and selenium.

As used herein, “cycloheteroalkyl” refers to a non-aromatic cycloalkyl group that contains at least one (e.g.. one, two, three-, four, or five) ring heteroatom selected from O, N, and S, and optionally contains one or more (e.g., one, two, or three) double or triple bonds, A cycloheteroalkyl group, as a whole, can have from 3 to 14 ring atoms and contains from 1 to 5 ring heteroatoms (e.g., from 3-6 ring atoms for a monocyclic cycloheteroalkyl group and from 7 to 14 ring atoms for a polycyclic cycloheteroalkyl group). The cycloheteroalkyl group can be covalently attached to the defined chemical structure at any heteroatom(s) or carbon atom(s) that results in a stable structure. One or more N or S atoms in a cycloheteroalkyl ring may be oxidized (e.g., morpholine N-oxide, thiomorpholine S-oxide, thiomorpholine S,S-dioxide). Cycloheteroalkyl groups can also contain one or more oxo groups, such as phthalimidyl, piperidonyl, oxazolidinonyl, 2,4(1H,3H)-dioxo-pyrimidinyl, pyridin-2(1H)-onyl, and the like. Examples of cycloheteroalkyl groups include, among others, morpholinyl, thiomorpholinyl, pyranyl, imidazolidinyl, imdazolinyl, oxazolidinyl, pyrazolidinyl, pyrazolinyl, pyrrolidinyl, pyrrolinyl, tetrahydrofuranyl, tetrahydrothienyl, piperidinyl, piperazinyl, azetidine, and the like.

As used herein, “aryl” refers to an aromatic monocyclic hydrocarbon ring system or a polycyclic ring system where at least one of the rings in the ring system is an aromatic hydrocarbon ring and any other aromatic rings in the ring system include only hydrocarbons. In some embodiments, a monocyclic aryl group can have from 6 to 14 carbon atoms and a polycyclic aryl group can have from 8 to 14 carbon atoms. The aryl group can be covalently attached to the defined chemical structure at any carbon atom(s) that result in a stable structure. In some embodiments, an aryl group can have only aromatic carbocyclic rings, e.g., phenyl, 1-naphthyl, 2-naphthyl, anthracenyl, phenanthrenyl groups, and the like. In other embodiments, an aryl group can be a polycyclic ring system in which at least one aromatic carbocyclic ring is fused (i.e., having a bond in common with) to one or more cycloalkyl or cycloheteroalkyl rings. Examples of such aryl groups include, among others, benzo derivatives of cyclopentane (i.e., an indanyl group, which is a 5,6-bicyclic cycloalkyllaromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system). Other examples of aryl groups include benzodioxanyl, benzodioxolyl, chromanyl, indolinyl groups, and the like.

As used herein, “heteroaryl” refers to an aromatic monocyclic ring system containing at least one ring heteroatom selected from O, N, and S or a polycyclic ring system where at least one of the rings in the ring system is aromatic and contains at least one ring heteroatom. A heteroaryl group, as a whole, can have from 5 to 14 ring atoms and contain 1-5 ring heteroatoms. In some embodiments, heteroaryl groups can include monocyclic heteroaryl rings fused to one or more aromatic carbocyclic rings, non-aromatic carbocyclic rings, or non-aromatic cycloheteroalkyl rings. The heteroaryl group can be covalently attached to the defined chemical structure at any heteroatom or carbon atom that results in a stable structure. Generally, heteroaryl rings do not contain O—O, S—S, or S—O bonds, However, one or more N or S atoms in a heteroaryl group can be oxidized (e.g., pyridine N-oxide, thiophene S-oxide, thiophene S,S-dioxide). Examples of such heteroaryl rings include pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl benzothiazolyl, benzisothiazolyl, benzisoxazolyl, benzoxadiazolyl, berizoxazotyl, cinnolinyl, 1H-indazolyl, 2H-indazolyl, indolizinyl, isobenzofuyl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienoxazolyl, thienoimidazolyl groups, and the like. Further examples of heteroaryl groups include 4,5,6,7-tetrahydroindolyl, tetrahydroquinolinyl, benzothienopyridinyl, benzofuropyridinyl groups, and the like.

The present invention includes all pharmaceutically acceptable isotopically-labeled compounds of the invention, i.e. compounds of Formula (I), wherein one or more atoms are replaced by atoms having the same atomic number, but an atomic mass or mass number different from the atomic mass or mass number usually found in nature.

Examples of isotopes suitable for inclusion in the compounds of the invention comprises isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of Formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of Formula (I) can generally be prepared by conventional techniques known to those skilled in the art or by processes analogous to those described in the accompanying Examples and Preparations using an appropriate isotopically-labeled reagents in place of the non-labeled reagent previously employed.

### EXAMPLES

Examples 1-3 illustrate the general procedure can be used to make 7-Cyclopentyl-2-(5-piperazin-1-yl-pyridin-2-ylamino)-7H-pyrrolo[2,3-d] pyrimidine-6-carboxylic acid dimethylamide (Compound A1), Additional methods for making the CDK4/6 inhibitors described herein can be found in WO Application No. PCT/EP09/060793, published as WO 2010/020675.

### Example 1

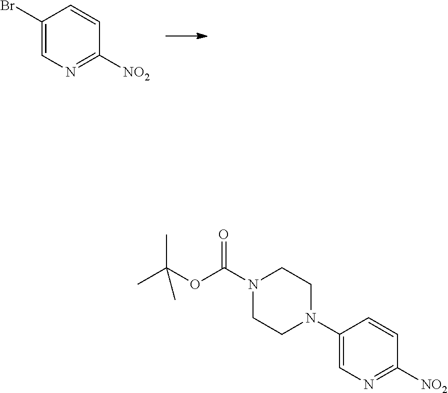 [SMILES: O=[N+]([O-])c1ccc(Br)cn1 ; CC(C)(C)OC(=O)N1CCN(c2ccc([N+](=O)[O-])nc2)CC1]

Nitrile analogues can be made by the following. To a stirred solution of 5-bromo-2-nitropyridine (4.93 g, 24.3 mmol) and piperazine-1 -carboxylic acid tent-butyl ester (4.97 g, 26.7 mmol) in CH₃CN (60 ml) is added DIPEA (4.65 mL, 26.7 mmol). The mixture is heated at reflux for 72 hours then cooled to room temperature and the precipitated product collected by filtration. The filtrate is concentrated and purified by flash column chromatography eluting with 30% EtOAc/petrol. The combined products are re-crystallized from EtOAc/petrol to give 4-(6-nitro-pyridin-3-yl)-piperazine-1-carboxylic acid tert-butyl ester, (4.50 g, 80% yield). MS(ESI) m/z 308 (M+H)⁺

### Example 2

A mixture of 5-[4-(2,2,2-trifluoro-ethyl)-piperazin-1-yl]-pyridin-2-ylamine (158 mg, 0.607 mmol), 2-chloro-7-cyclopentyl-7H-pyrrolo[2,3-d]pyrimidine-6-carboxylic acid dimethylamide (118 mg, 0.405 mmol), Pd₂(dba)₃ (18.5 mg, 0.020 mmol), BINAP (25 mg, 0.040 mmol) and sodiurn-tert-butoxide (70 mg, 0.728 mmol) in dioxane (3.5 mL) is degassed and heated to 100° C. for 1 h in a CEM Discover microwave, The reaction mixture is partitioned between dichloromethane and saturated NaHCO₃ solution. The organic layer is separated and the aqueous layer extracted with further dichloromethane. The combined organics are ished with brine, dried (MgSO₄), filtered and concentrated. The crude product is purified using silica gel chromatography (0 to 10% methanol/dichloromethane) to give 7-cyclopentyl-2-{5-[4-(2,2,2-trifluoro-ethyl)-piperazin-1-yl]-pyridin-2-ylamino}-7H-pyrrolo[2,3-d]pyrimidine-6-carboxylic acid dimethylamide, which is purified further by trituration with acetonitrile (115 mg, 55%). MS(ESI) m/z 517.2 (M+H)⁺ (method A).

¹H NMR (400 MHz, Me-d₃-OD): 8.72 (1H, s), 8.24 (1H, d), 7.98 (1H, d), 7.50 (1H, dd), 6.62 (1H, s), 4.81-4.72 (1H, m), 3.27-3.09 (12H, m), 2.89 (4H, t), 2.61-2.49 (2H, m), 2.16-2.01 (4H, m), 1.81-1.69 (2H, m).

### Example 3

### 7-Cyclopentyl-2-(5-piperazin-1-yl-pyridin-2-ylamino)-7H-pyrrolo[2,3-d]pyrimidine-6-carboxylic acid dimethylamide

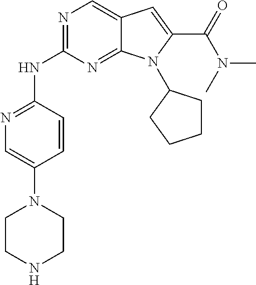 [SMILES: CN(C)C(=O)c1cc2cnc(Nc3ccc(N4CCNCC4)cn3)nc2n1C1CCCC1]

Following Buchwald Method of Example 2, then General Procedure of Example 1, 2-chloro-7-cyclopentyl-7H-pyrrolo[2,3-d]pyrimidine-6-carboxylic acid dimethylamide (300 mg, 1.02 mmol) and 5-piperazin-1-yl-pyridin-2-ylamine (314 mg, 1.13 mmol) gave 7-cyclopentyl-2-(5-piperazin-1-yl-pyridin-2-ylamino)-7H-pyrrolo[2,3-d]pyrimidine-6-carboxylic acid dimethylamide (142 mg, 36%). MS(ESI) m/z 435.3 (M+H)⁺

### Example 4

The Cell Titer-Glo Luminescent Cell Viability Assay (Promega# G7572) generates a luminescent signal that is proportional to the number of metabolically active cells present in a reaction, based on the quantitation of ATP. Cell Titer-Glo Reagent was prepared by thawing a vial of Cell Titer-Glo Buffer in a 37° C. water bath. The entire bottle of buffer was then added to the bottle of lyophilized Cell Titer-Glo Substrate provided in the kit. Lyophilized substrate was allowed to dissolve; the solution was then mixed by inversion and was ready for use. Jeko-1 cells were diluted to a density of 200,000 cells/mL and cultured in T250 flask. Before treatment (time 0), 3×100 micro liter aliquots were removed and placed into a black 96 well plate with clear bottom (Costar#3904). 50 uL of CTG reagent was added to each well. The plate was placed on an Orbital Shaker, protected from light and incubated using setting 4 for 30 minutes at RT. The plate was then read using the Envision Luminometer, and results exported. The cells remaining in the T250 flasks were either left untreated or treated with single agents or in combinations. The concentration of CDK4/6 inhibitor used was 100 nM and those of mTOR inhibitor used were 1, 2.5 and 5 nM. Plates were allowed to incubate for 72 hrs at 37° C. and 5% CO₂. After 72 hrs, 3×100 uL aliquots were removed and subjected to CTG as described above. Results were exported and analyzed using Microsoft Excel. The percentage of viable cells as compared to control growth was calculated using following equation:

If A>B, then 100×((A−B)/(C−B)), if not then 100−(A−B/B)

Where:

A is the CTG read under treatment condition

B is the CTG read for Time 0 cells

C is CTG read for 72 hr untreated cells

### Example 5

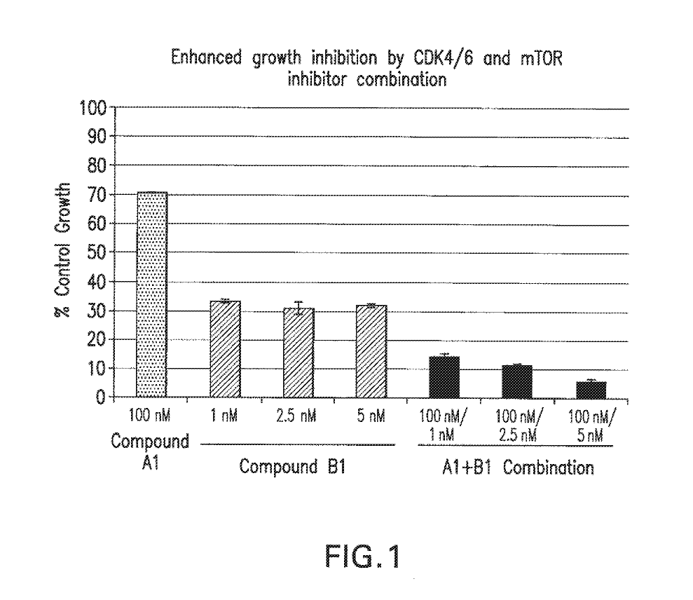

To evaluate whether the CDK4/6 and mTOR inhibitor combination leads to more pronounced growth inhibition compared to the growth inhibitions observed with single agents, Jeko-1 mantle cell lymphoma cells were treated with 100 nM of CDK4/6 inhibitor, 1, 2.5 and 5 mM of mTOR inhibitor and the combinations of the two inhibitors, as shown in FIG. 1. Growth inhibitions are measured using the CellTiter-Glo kit of Example 4. % growth of the treated cells, compared to the vehicle control, was obtained. As shown in FIG. 1, the treatment with 100 nM of CDK4/6 inhibitor led to 70% cell growth compared to the vehicle control, while the treatments with mTOR inhibitor alone led to approximately 30% growth at all three concentrations tested. Notably, the combinations of CDK4/6 and mTOR inhibitor led to much more pronounced growth inhibitions at all combinations tested. For example, less than 10% cell growth was observed for the 100 nM/5 nM CDK4/6 and mTOR inhibitor combination. This shows that CDK4/6 and mTOR inhibitor combination induces higher amounts of cell growth inhibitions, when evaluated against the single agent activities of the individual compound.

### Example 6

To determine if CDK4/6 and mTOR inhibitor combinations resulted in synergistic growth inhibitions, we generated isobolograms, where we compared the actual growth inhibition values in combinations, to 25, 50 and 75% growth inhibitions predicted for additivity (Tallarida R J (2006) An overview of drug combination analysis with isobolograms. Journal of Pharmacology and Experimental Therapeutics; 319 (1):1-7). Briefly, 9 titrating concentration points including 0 nM that yielded growth inhibition values that ranged from 0 to 100% as single agents were determined for both CDK4/6 and mTOR inhibitor. In a 96 well plate, the 9 concentration points for each agent were mixed in a matrix format, generating 81 combinations. This plate was used to treat Jeko-1 cells, and the resulting growth inhibition values were used to generate 10₅₀ values for the single agents and combinations. Graph was generated with CDK4/6 inhibitor concentrations shown on the y-axis and mTOR inhibitor concentrations shown on the x-axis. A straight line connecting the CDK4/6 inhibitor and the mTOR inhibitor IC₅₀ values represented growth inhibitions that were strictly additive for the combinations. Plots placed below the line of additivity (more growth inhibition) represented synergistic growth inhibitions, while plots above the line of additivity (less growth inhibition) represented antagonistic growth inhibitions.

### Example 7

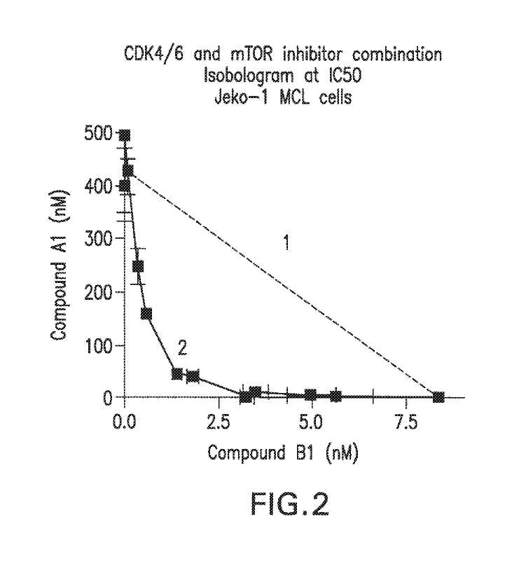

To evaluate whether the cell growth inhibition by the CDK4/6 and mTOR inhibitor combination is synergistic, we measured the single agent and combination activities in Jeko-1 cells and analyzed them using the isolobologram analysis prepared according to Example 6. Briefly, the single agent activities of CDK4/6 and mTOR inhibitors were measured to determine 9 titrating concentration points that would give 0 to 100% growth inhibitions for each agent. In a matrix format, all possible combinations for the 9 concentration points a each inhibitor were co-administered to Jeko-1 cells and the observed growth inhibitions were recorded. The concentrations that gave 50% growth inhibitions were then calculated for each compound and the combinations, and used to generate the graph shown in FIG. 2. Axis X and Y represent mTOR and CDK4/6 inhibitor concentrations, respectively. Line 1 represents the growth inhibitions predicted for additivity, when considering 50% growth inhibitions. Line 2 is the plot generated for the observed combinations concentrations that gave the 50% growth inhibitions, and it is profoundly placed below the line of additivity, suggesting a strong synergy for the growth inhibition. In summary, the CDK4/6 and mTOR inhibitor combination inhibited cell growth in synergistic manner in Jeko-1 mantle cell lymphoma cells.

### Example 8

The synergistic effect in a breast cancer cell line MDA-MB453 by the CDK4/6 and mTOR inhibitor combination was also analyzed using an isoloblogram analysis as described in Example 7 above. Also in accordance with Example 7, the CDK4/6 and mTOR inhibitor combination inhibited cell growth in synergistic manner in MDA-MB453 breast cancer cells.

### Example 9

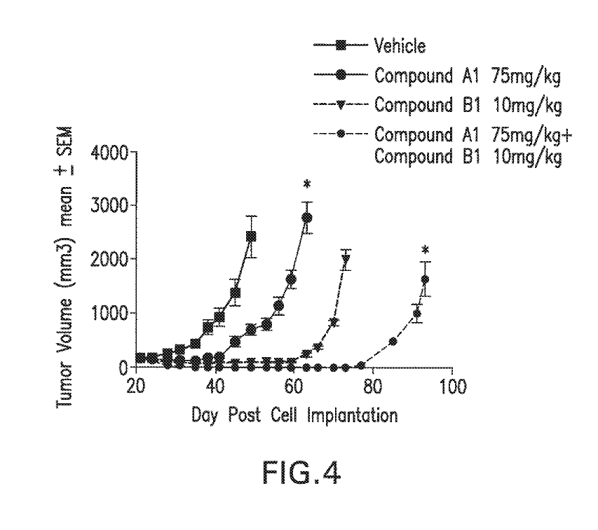

A Delco-1 xenograft model was used to measure anti-tumor activity in a 35 day treatment period of Compound A1, Compound B1, and the combination of Compounds A1 and B1. Significant antitumor activity was observed. When dosing was stopped and tumors were allowed to re-grow, the combination of Compounds A1 and B1 significantly delayed tumor growth by 20 days. In this model, both Compound A1 and Compound B1 had anti-tumor activity. However, the combination of Compounds A1 and B1 significantly extended tumor growth delay when treatment was stopped. See FIG. 4.

### Example 10

The PANC-1 pancreatic carcinomas used for implantation were maintained by serial engraftment in nude mice. To initiate tumor growth, a 1 mm3 fragment was implanted subcutaneously in the right flank of each test animal. Tumors were monitored twice weekly and then daily as their mean volume approached 100-150 mm3. Twenty two days after tumor cell implantation, on D1 of the study, the animals were sorted into four groups of ten mice, with individual tumor sizes of 108-221 mm3 and group mean tumor sizes of 150-153 mm3. Tumor size, in mm3, was calculated from:

Tumor Volume=(w2×l)/2

where w=width and l=length, in mm, of the tumor. Tumor weight can be estimated with the assumption that 1 mg is equivalent to 1 mm3 of tumor volume.

Group 1 mice received the Compound A1 and Compound B1 vehicles, and served as controls for all analyses. Groups 2 and 3 received monotherapies with 250 mg/kg qd, po×21 days of Compound A1 or 10 mg/kg qd, po×21 days of Compound B1. Group 4 received the combination therapy of Compound A1 and Compound B1.

Each animal was euthanized when tumor volume reached 1200 mm3, or on the last day of the study (D55). For each animal whose tumor reached the endpoint volume, the time to endpoint. (TTE) was calculated by the following equation:

TTT=(log₁₀(endpoint volume)−b)/m

Where TTE is expressed in days, endpoint volume is in mm3, b is the intercept, and m is the slope of the line obtained by linear regression of a log-transformed tumor growth data set. The data set is comprised of the first observation that exceeded the study endpoint volume and the three consecutive observations that immediately preceded the attainment of the endpoint volume. The calculated TTE is usually less than the day on which an animal is euthanized for tumor size. An animal with a tumor that did not reach the endpoint is assigned a TIE value equal to the last day. An animal classified as having died from TR causes or non-treatment-related metastasis (NTRm) is assigned a TTE value equal to the day of death. An animal classified as having died from NTR causes is excluded from TTE calculations.

Treatment efficacy was determined from tumor growth delay (TGD), which is defined as the increase in the median TTE for a treatment group compared with the control group: TGD=T−C, expressed in days, or as a percentage of the median TTE of the control group: % TGD=[(T−C)/C]×100, where: T=median TTE for a treatment group, C=median TTE for the designated control group.

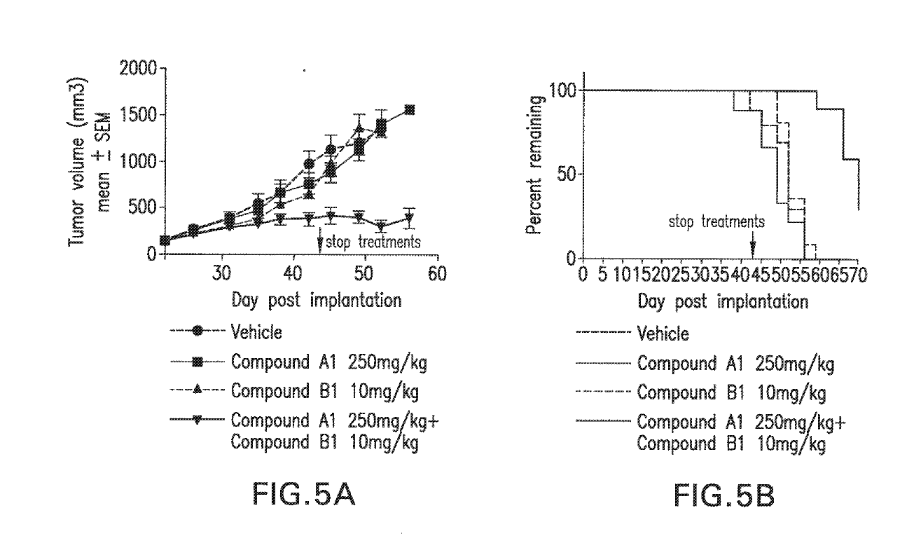

These studies demonstrate that neither Compound A1 nor Compound B1 had significant anti-tumor activity in the PANC-1 xenograft model. However, the combination of Compounds A1 and B1 resulted in tumor stasis (FIG. 5A) and significantly delayed tumor regrowth by 18 days (FIG. 5B).

### Example 11

Potential synergistic interactions between CDK4/6 and mTOR inhibitor combinations were assessed relative to the Loewe additivity model using CHALICE software, via a synergy score calculated from the differences between the observed and Loewe model values across the response matrix. Briefly, 9 titrating concentration ranging from 10 uM diluted serially three folds for CDK4/6 inhibitors and 0.1 uM diluted serially 3 folds for the mTOR inhibitors, including 0 uM, were used in a 96 well plate, the 9 concentration points for each agent were mixed in a matrix format, generating 81 combinations. This plate was used to treat Jeko-1 cells, and the resulting inhibition values were used by CHALICE software to generate inhibition and ADD Excess Inhibition matrices as well as the isobolograms. A more detailed explanation of the technique and calculation can be found in Lehar et al. “Synergistic drug combinations improve therapeutic selectivity”, Nat. Biotechnol. 2009, July; 27(7), 659-666, which is hereby incorporated by reference.

Inhibition matrix shows the actual inhibition observed by the CTG assay at the respective concentrations of the compounds. ADD Excess inhibition shows the excess inhibition observed over the inhibition predicted by the Loewe additivity model. In addition to the matrices, one can use isobolograms to observe synergy. The inhibition level for each isobologram was chosen manually so as to observe the best synergistic effects. Isobologram was generated with CDK4/6 inhibitor concentrations shown on the y-axis and mTOR inhibitor concentrations shown on the x-axis. A straight line connecting the CDK4/6 inhibitor and the mTOR inhibitor concentrations which produce the chosen level of inhibition represented growth inhibitions that were strictly additive for the combinations. Plots placed below the line of additivity (more growth inhibition) represented synergistic growth inhibitions, while plots above the line of additivity (less growth inhibition) represented antagonistic growth inhibitions.

Synergic interaction between the following pairs of CDK4/6 inhibitor and the mTOR inhibitor combination were studied, the synergy scores, and the corresponding figure illustrations are listed below:

| CDK4/6 inhibitor | mTOR inhibitor | Synergy Score | FIG. |
|---|---|---|---|
| Compound A1 | Compound B1 | 4.92 | 6A-6C |
| Compound A1 | Compound B2 | 7.77 | 7A-7C |
| Compound A4 | Compound B1 | 7.16 | 8A-8C |
| Compound A2 | Compound B1 | 3.76 | 9A-9C |
| Compound A3 | Compound B1 | 7.1 | 10A-10C |
| Compound A6 | Compound B1 | 6.41 | 11A-11C |
| Compound A5 | Compound B1 | 4.04 | 12A-12C |
| Compound A4 | Compound B2 | 5.73 | 13A-13C |
| Compound A2 | Compound B2 | 4.57 | 14A-14C |
| Compound A3 | Compound B2 | 6.85 | 15A-15C |
| Compound A6 | Compound B2 | 3.24 | 16A-16C |
| Compound A5 | Compound B2 | 5.86 | 17A-17C |

1. A combination comprising a first agent that is a cyclin dependent kinase 4 or cyclin dependent kinase 6 (CDK4/6) inhibitor and a second agent that is an mTOR inhibitor, wherein the first agent is 7-Cyclopentyl-2-(5-piperazin-1-yl-pyridin-2-ylamino)-7H-pyrrolo[2,3-d]pyrimidine-6-carboxylic acid dimethylamide or a pharmaceutically acceptable salt thereof.

2. The combination of claim 1, wherein the second agent is selected from the group consisting of rapamycin (AY-22989), everolimus, CCI-779, AP-23573, MK-8669, AZD-8055, Ku-0063794, OSI-027, WYE-125132.

3. The combination of claim 2, wherein the second agent is everolimus.

4. The combination of claim 1, wherein the first agent and the second agent are in a combined dosage form.

5. The combination of claim 1 wherein the first agent and the second agent are in separate dosage forms.

6. A method of treating cancer comprising administering a first agent that is a cyclin dependent kinase 4 or cyclin dependent kinase 6 (CDK4/6) inhibitor and a second agent that is an mTOR inhibitor wherein the first agent is 7-Cyclopentyl-2-(5-piperazin-1-yl-pyridin-2-ylamino)-7H-pyrrolo[2,3-d]pyrimidine-6-carboxylic acid dimethylamide or a pharmaceutically acceptable salt thereof.

7. The method of claim6 wherein the cancer is dependent on the CDK4, CDK6 or mTOR pathway.

8. The method of claim 7 wherein the cancer is a solid tumor cancer.

9. The method of claim 6, wherein the cancer is pancreatic cancer, breast cancer, mantle cell lymphoma, non-small cell lung cancer, melanoma, colon cancer, esophageal cancer, liposarcoma, multiple myeloma, T-cell leukemia, renal cell carcinoma, gastric cancer, renal cell carcinoma, glioblastoma, hepatocellular carcinoma, gastric cancer, lung cancer or colon cancer.

10. The method of claim herein the cancer is pancreatic cancer, breast cancer, or mantle cell lyomphoma.

11. The method of claim 6, wherein the cancer is a lymphoma.

12. The method of claim 6 wherein the first agent and the second agent are administered in a combined dosage form.

13. The method of claim 6 wherein the first agent and the second agent are administered in separate dosage forms.

14. The method of claim 6 wherein the second agent is everolimus.
